SEQUENCE LISTING (38 sequences)

SEQ ID NO: 1 - DNA, 996 bp, Arabidopsis thaliana
  CDS (18)..(638)
gtagttctaaaaaattaatggtgtcgtcaaagaaacccaaggagaagaaggcgaggagcgatgtcgtcgtcaataaagcgagtggtcggagtaaacgcagctccggttccagaacgaagaagacgtcgaacaaggttaacattgtgaagaagaagccggagatttacgagatctcagaatcatcgagcagtgactctgtggaagaagcaataagaggcgatgaggcgaagaaaagtaacggcgtcgtgagcaagaggggtaacggaaagagtgtaggaattccgacgaagacgagtaaaaatcgagaagaggacgatggaggcgcggaagatgctaagatcaagtttccgatgaatcggattcggcggatcatgagaagcgataattctgctcctcagattatgcaggatgctgtatttcttgtcaacaaagccacggagatgttcattgagcggttttctgaagaagcttatgatagttccgtcaaggacaaaaagaaattcatccactacaaacacctctcatccgtagtgagtaacgaccagagatacgagttccttgcagatagtgttcccgagaaacttaaagcagaggccgcgttggaggaatgggaaagaggcatgacagatgcaggctgaaataaatccggttggaatcgaactgaaccatttggattcaaatttgtgtccctgtcctggtttatacttcataggttccatccgcccgatcttcacctttcttattaaccactactgagattgcacatatttgatttctagaatgtatacattttttttcttgtttcaagttaaaatttgtaagagttgtcggaattctaatatctttctttagtatctatatatacatttttgcagccattttgtgaaataaattttgtagttggacttatttacaatggctgccactggattttgatgtttgtatttgataattagaaagaaaatcttcgaattaaatatttgacatttaacaatc

SEQ ID NO: 2 - protein, 206 aa, Arabidopsis thaliana
MVSSKKPKEKKARSDVVVNKASGRSKRSSGSRTKKTSNKVNIVKKKPEIYEISESSSSDSVEEAIRGDEAKKSNGVVSKRGNGKSVGIPTKTSKNREEDDGGAEDAKIKFPMNRIRRIMRSDNSAPQIMQDAVFLVNKATEMFIERFSEEAYDSSVKDKKKFIHYKHLSSVVSNDQRYEFLADSVPEKLKAEAALEEWERGMTDAG

SEQ ID NO: 3 - DNA, 1581 bp, Oryza sativa
  CDS (111)..(1022)
ttgagtttcagcatcagcgcccccaaaccaaataattttgaactctccggactccggccgccgccttccagaggaacctcgcggctgccggtgccggtgccggagaagacatggccgggaagaagaaggccctaacaaatcccgcctctccctccgcctccgcctccgcttccacccctaagaaatccaccgccacctccaaggaccgctccacgccgaaaccccgcaaaaaccccaaccccaaggaggaggcaccaccgccgccgcccgccaacaacaagcgcctcaacccccaagggggatccaaccgtaagaagaaggccgatgcggggacgccctccaagaagccgaagcgccagccgccggagcccaaacctcggaagcacaagggggccaagagcgagaagccgcaccgggtttccggcgaaggggagaagcccacgcccacgaagaagaagaagaagaaggagagcagcaaggagcccaagcgcgagaagcagcaagcgtccgcgcctatgtcgactccctcgaagaagaacaaggaagccaaacgcgacacaggaggagcggggaagcccacgcccaccaagaggaagctcggcgacgtggatcccccacaggagcgaccgtccggtgaaggccaggcaagctctcccacgcccgccaagaagcggaaggacaaggcggcggcggcggaggcggttgccgaccacggcgccggcagcttcccgatggctcgggtgcggcagataatgcgggccgaggacgccaccatccgcccctccaatgaggctgtcttcctcatcaacaaggccaccgagatattcttgaagaggtttgcagacgatgcttatcgaaatgctttgaaggatcggaagaagtcaattgtgtatgataacctttcaacagcagtgtgcaaccagaaaagatacaagtttctctcagattttgttccacagaaagttacagctgaagatgctttgaaggctccagttagcagccaagtgaaccaaccacaataagttgctagtgctccgcgaggtatgctgctatgtttttactacttcctagcttttttaaggagcgaattactggaaattagttttccatgttgtgaaagttccagcattcagctgctccatcctgtagggaggtcatcctttcacgagttatctcctgggagggttctgcaattgtaaacgccagctgtgcagctcatgttgccctcaggactggcatcttgcgacatgttcaatatgattctgatcagaatcggaggcttaaaagaagattataaggggaaacctgagtaatctacatatgaaagaagcatgatgaaactttttgtcatgagaatcacttgaaatatgcttgcttagagcctcaaatcaggatcacctttttggctgtgtaagctgtgggttgtgtgaggtggatgtagcttaggctaaatgttactgtgtatttttttttagataatacgttactagtagtatacatttgtgagttctgacttgttcatgtactagttcattacatatattttcgcaggtgcatgctttaccaattacttgctggtgc

SEQ ID NO: 4 - protein, 303 aa, Oryza sativa
MAGKKKALTNPASPSASASASTPKKSTATSKDRSTPKPRKNPNPKEEAPPPPPANNKRLNPQGGSNRKKKADAGTPSKKPKRQPPEPKPRKHKGAKSEKPHRVSGEGEKPTPTKKKKKKESSKEPKREKQQASAPMSTPSKKNKEAKRDTGGAGKPTPTKRKLGDVDPPQERPSGEGQASSPTPAKKRKDKAAAAEAVADHGAGSFPMARVRQIMRAEDATIRPSNEAVFLINKATEIFLKRFADDAYRNALKDRKKSIVYDNLSTAVCNQKRYKFLSDFVPQKVTAEDALKAPVSSQVNQPQ

SEQ ID NO: 5 - DNA, 908 bp, Glycine max
  CDS (98)..(658)
gtgtgagcacgcttcttcttggagacctgcttccgacggacgctgaaagtgaaacgcaaaatcccgtgacggagtgcttccactgcactgtcatacaatggcttcctccaacactcccaaacccgaaaataagaagagtaccaaaaaatccgaaatctccaaagccgaaaagaagaaaaccaaaaacgctgaaatccccaaaaccgatggcaaaacgaagaagaacaaagaaatctcacaagaggagaacaagaagaagattaagaaggccaagctcagcaacggcacttcaaaacagcgagacgagggatccaaaaagggagtagcagcagaaggaaacggagaagaggcgaaaatgaacgtgtttccgatgaatcgcatcaggacgatgattaagggcgaagatcccgaaatgcgtgtttctcaggaagcattgtttgccatcaacaacactgtggaaaagttccttgagcagttcacgcaggacgcttatgccttttgtgctcaggaccgcaagaaatgcctcagctatgatcacctagcacatgttgtaagtaagcaaaggagatatgactttctctctgattttgttcctgagagagtaaaagctgaggatgcattaagagagagaagtgcagcaggaaaaggaggaagttaaaaaatctaaagcaagctagtggagaatataagcttatactttatttaatcatgtaacattttgtaatggaaacatgcatcatgatgagtaatgctgtactttaagtttccatattgtatttattttagtaatcctattcccgtaagtgtatatgatttttcagtgactatgcatgtgcttgactctgcttgggtagagcatggtgatggtgtcaaatttcttatgttgaaaatctgaagttaattttccc

SEQ ID NO: 6 - protein, 186 aa, Glycine max
MASSNTPKPENKKSTKKSEISKAEKKKTKNAEIPKTDGKTKKNKEISQEENKKKIKKAKLSNGTSKQRDEGSKKGVAAEGNGEEAKMNVFPMNRIRTMIKGEDPEMRVSQEALFAINNTVEKFLEQFTQDAYAFCAQDRKKCLSYDHLAHVVSKQRRYDFLSDFVPERVKAEDALRERSAAGKGGS

SEQ ID NO: 7 - DNA, 812 bp, Glycine max
  CDS (93)..(650)
gggtattgcgcacgcttcttctttggagactgtttccgacagagggtgaaactgaaactcccgtgtcggggtgtttacactgcactgtcacaatgacttcctccaactctccgaaacctgaaaagaaggaaaagaagaaaaacgctgaaatccccaaaatcgaaaagaagaaaaccaaaagcgctgaaatccctctaaccgatggcaaaacgaagagggatagagaaatcgccaaagaggagaacaagaagaagactaagaaacccaagctcagcaatggcacttcaaaacagcgagacgagggatccaaaaagggagtagcagaaggaaaaggagaagaggggaaaatgaacgtgtttccgatgaatcgcatcagaacgatgattaagggcgaagatcccgatatgcgtgtttctcaggaagcattgttggccatcaacaacgctgtggagaagttccttgagcaattctcgcaggaagcttatgctttttgtgttcgggaccgcaagaaatgcctcagctacgatcacctagcacatgttgtaagtaagcaaaggagatatgactttctctctgattttgttcctgagagagtgaaagctgaggatgcattaagagagagaagtgcagcaggaacaggaggacactaaaaaatctaaagtaagctagtggagaatataaacttatacttttatttaatcatgtaacatttttgtaatggaaacatgcatcatgatgagtaatgctgtactttatgtttccatattgtatttattttagtaatcctattgccgtaagtgtatatgattttt

SEQ ID NO: 8 - protein, 185 aa, Glycine max
MTSSNSPKPEKKEKKKNAEIPKIEKKKTKSAEIPLTDGKTKRDREIAKEENKKKTKKPKLSNGTSKQRDEGSKKGVAEGKGEEGKMNVFPMNRIRTMIKGEDPDMRVSQEALLAINNAVEKFLEQFSQEAYAFCVRDRKKCLSYDHLAHVVSKQRRYDFLSDFVPERVKAEDALRERSAAGTGGH

SEQ ID NO: 9 - protein, 19 aa, Arabidopsis thaliana
RYEFLADSVPEKLKAEAAL

SEQ ID NO: 10 - protein, 19 aa, Oryza sativa
RYKFLSDFVPQKVTAEDAL

SEQ ID NO: 11 - protein, 19 aa, Glycine max
RYDFLSDFVPERVKAEDAL

SEQ ID NO: 12 - protein, 19 aa, Glycine max
RYDFLSDFVPERVKAEDAL

SEQ ID NO: 13 - protein, 19 aa, Physcomitrella patens
RLEFLSDIVPVRIPAAAAL

SEQ ID NO: 14 - DNA, 57 bp, Arabidopsis thaliana
agatacgagttccttgcagatagtgttcccgagaaacttaaagcagaggccgcgttg

SEQ ID NO: 15 - DNA, 57 bp, Oryza sativa
agatacaagtttctctcagattttgttccacagaaagttacagctgaagatgctttg

SEQ ID NO: 16 - DNA, 57 bp, Glycine max
agatatgactttctctctgattttgttcctgagagagtaaaagctgaggatgcatta

SEQ ID NO: 17 - DNA, 57 bp, Glycine max
agatatgactttctctctgattttgttcctgagagagtgaaagctgaggatgcatta

SEQ ID NO: 18 - DNA, 27 bp, Artificial Sequence
  Primer
ccatcgatacatggtgtcgtcaaagaa

SEQ ID NO: 19 - DNA, 26 bp, Artificial Sequence
  Primer
atctcgagtcagcctgcatctgtcat

SEQ ID NO: 20 - DNA, 25 bp, Artificial Sequence
  Primer
cgtctagaatggtgtcgtcaaagaa

SEQ ID NO: 21 - DNA, 26 bp, Artificial Sequence
  Primer
atatcgattccgcctgcatctgtcat

SEQ ID NO: 22 - DNA, 26 bp, Artificial Sequence
  Primer
gctctagagatggtgtcgtcaaagaa

SEQ ID NO: 23 - DNA, 26 bp, Artificial Sequence
  Primer
atctcgagtcagcctgcatctgtcat

SEQ ID NO: 24 - protein, 86 aa, Arabidopsis thaliana
FPMNRIRRIMRSDNSAPQIMQDAVFLVNKATEMFIERFSEEAYDSSVKDKKKFIHYKHLSSVVSNDQRYEFLADSVPEKLKAEAAL

SEQ ID NO: 25 - protein, 86 aa, Oryza sativa
FPMARVRQIMRAEDATIRPSNEAVFLINKATEIFLKRFADDAYRNALKDRKKSIVYDNLSTAVCNQKRYKFLSDFVPQKVTAEDAL

SEQ ID NO: 26 - protein, 86 aa, Glycine max
FPMNRIRTMIKGEDPEMRVSQEALFAINNTVEKFLEQFTQDAYAFCAQDRKKCLSYDHLAHVVSKQRRYDFLSDFVPERVKAEDAL

SEQ ID NO: 27 - protein, 86 aa, Glycine max
FPMNRIRTMIKGEDPDMRVSQEALLAINNAVEKFLEQFSQEAYAFCVRDRKKCLSYDHLAHVVSKQRRYDFLSDFVPERVKAEDAL

SEQ ID NO: 28 - protein, 86 aa, Physcomitrella patens
FPISRVRRLVKSEGDIQWVGVEAGFLIAKATEIFLEKLVEDAFERMQGNGQASILYPHLSSHVASSERLEFLSDIVPVRIPAAAAL

SEQ ID NO: 29 - DNA, 1722 bp, Oryza sativa
gcctttccttccgatctctctccctctctctcttcttcttcttcttccttccctctcaacccgacgacccacgcgaagcgaactctcgcgcgagacgagagtagtaaaccctagaaacctagaggagatccccaccaccaccatgacggtggatcagaggacgacggcgaaggcgatcatgccgccggtggagatgccgcccgtccagcccggaaggaaaaagcgaccacggagatcacgcgatggacctacttcagttgcagagaccatcaagcggtgggccgagctcaacaatcagcaggagcttgatccacagggtccaaagaaggcaaggaaggcacctgcaaagggttcaaagaagggctgcatgaaggggaaaggaggaccggagaatacacgttgtgacttccgtggtgtgaggcaacgtacctggggcaagtgggttgctgaaattcgggagccgaatcagcaaagtagactctggttggggaccttcccaactgccgaagctgcagcttgtgcttatgacgaggcagccagagcaatgtatggtccaatggctcgcactaattttggccagcatcatgcccctgctgcttccgttcaggttgcactagcagctgtcaaatgtgctttacctggtggtggcttaacagcaagcaagtctagaacatccactcagggtgcatcagcagatgttcaagatgttttaactggtggcttatcagcatgcgagtccactacaacaacaattaataatcaatctgatgtcgtctctaccttacataagccagaagaggtttctgagatctctagtccactgagagctccaccagctgtcctggaagatggttctaatgaagacaaggctgaatcggttacctatgatgagaacattgtcagccagcagcgtgcccctcctgaagccgaggctagtaatggaagaggcgaggaggtctttgagcctctggaacctattgccagcctaccagaggaccaaggagattattgttttgatattgatgagatgctgagaatgatggaagctgaccctacgaacgagggtttgtggaaaggcgacaaagatggatcagacgccatcctggagcttggccaggatgaacctttctactacgaaggggttgatccaggcatgctggacaacttgctcaggtctgatgagccagcatggttattggcagatcctgcgatgttcatctccggtggcttcgaagatgactctcagttctttgagggcttgtgatttccccttggcggcagccggccatactaaaattttctggtgctttggtcggctagctcctgcacatcgccctcaggatcagcaagagaaacactggaccggattgggttcgttggtggaactggatgagcatctagtagctaaggaaaaaagatccttttatttagttctgtaggcaatggaactccttgagaactccgtttcagtgtttgttaatttgataacgcttgcttgtttgtgtgtgtatatcgatctcttttgaagcaatgagaaaaaaaaaaggactgaagaaaatgtgtatatattccaagcgttcttcagcctttcttagccttcatattttacctatgcacgtgggatgttgcagttttagagcttgtgagccttctctaaaaccggggattaaaatgcgactaggcacgatatgttcaatctaaaccgaactccctagggtgtat

SEQ ID NO: 30 - DNA, 29 bp, Artificial Sequence
  Primer
tagaattcatgacggtggatcagaggacg

SEQ ID NO: 31 - DNA, 27 bp, Artificial Sequence
  Primer
atggatccggccaaaattagtgcgagc

SEQ ID NO: 32 - DNA, 1901 bp, Glycine max
agagatttttctgaatccgctatagccataactcttcacgaacaagaactctactattactattaatcaaccaaaatctctcttcactccaaacagaacacactagcgagaaaaaaagtgataagcccaaaaactctgcgttctctcacaaattaaacagcgtcactatcgcatagattgtgaattcagtgattgagttttgcggtgtactgtgttgcgaagtctgtgtatcagatttgtggacatgggtgcttatgatcaagtttctcttaagccattggattcttctagaaagaggaaaagtaggagcagagggtatgggactggatccgtggctgagactattgcaaagtggaaggaatacaacgaacatctttattctggcaaagatgatagtagaacaactcgaaaggcaccggctaaaggttcgaagaaagggtgcatgaaagggaagggaggacctcaaaactctcagtgtaactacagaggagttaggcagaggacatgggggaaatgggttggtgagattagggagcccaatagaggaagcaggctttggttgggtaccttctcttctgcccaggaagctgctcttgcctatgatgaagctgctagagctatgtatggtccttgtgcacgcctcaattttcccggcatcacagattatgcttcttttaaggaatcgttgaaggaatctccgatggccgcatcgtcctcttgttcttcggcagaaactgcaacatctgacactactactacatccaaccaatcggaggtttgtgcagctgaggatgttaaggagaatcctcgacttgtcaatgtgaatgataaggttaacgattgtcataaggcttatgaagctgcctcaccaactagcagaatgaagcaagagcctaaggatgaggctgtggatcacatggtccccggggctgggaaaattctagatgtcagaccagaaggaacacatgatgccgggcaggttgcagaggatgtaaacaaagatcagatggacttgccatggattgatggctttgattttagtgacaattacttgaacaggttttccacggatgagttatttcaggtggatgaacttttggggcttatagataataacccaattgatgagtctgcgttgatgcaaagtttggattttggacaaatgggttttcctggagatggtaatcctcaggtggatgatacgctttcaagctttatttatcagttgcaaaatccagatgccaagttgttgggaagtttgccccatatggagcagacaccttcaggttttgattatggattagatttcttaaaaacagtggagtcaggggattataatggtggaggggaagaaccgcgatttcttaatttggatgatgatctgaaccctgattcaaagggcatgcaagcaaggaaggatgactagagaaggcgacgtgcataagtctatcatctgcctcattttcaactggttcgagcatctgctagtaatctgtctcttaggttgttgtcccctttttagctatatacaggtgcataagaggaatacaactatacaactaatacaagaaatttgatttgtttatgttcttttaatatgctaattctctgtaagattttttaaaatggagaatttagctgtgacaatatttgttaattctttttacttacatgttttttgggattcaaattggactgcctttaactacataggtggagctgaggagtagactgtttgaagtcgtttggctgactatagttgagcactgatttggatacaaaatttctttgttatgtaccatggagaactattatatctcgagtatattatatcgttgctcactttttgtgtataaaaactgaacaagtagtggaatgtatatatatataataactattct

SEQ ID NO: 33 - DNA, 31 bp, Artificial Sequence
  Primer
gcgaattcatgggtgcttatgatcaagtttc

SEQ ID NO: 34 - DNA, 28 bp, Artificial Sequence
  Primer
atggatcctttgggaaaattgaggcgtg

SEQ ID NO: 35 - DNA, 29 bp, Artificial Sequence
  Primer
taatcgatatgacggtggatcagaggacg

SEQ ID NO: 36 - DNA, 29 bp, Artificial Sequence
  Primer
atctcgagtcccaagccctcaaagaactg

SEQ ID NO: 37 - DNA, 31 bp, Artificial Sequence
  Primer
gcatcgatatgggtgcttatgatcaagtttc

SEQ ID NO: 38 - DNA, 28 bp, Artificial Sequence
  Primer
atctcgagctagccacccttccttgctt

(19) United States
(12) Patent Application Publication
(10) Pub. No.: US 2015/0128304 A1
(43) Pub. Date: May 7, 2015

(54) Plant Body Showing Improved Resistance Against Environmental Stress and Method for Producing Same

(71) Applicant: The University of Tokyo, Tokyo (JP)

(72) Inventors: Kazuko Shinozaki, Tokyo (JP); Hikaru Sato, Tokyo (JP)

(73) Assignee: The University of Tokyo, Tokyo (JP)

(21) Appl. No.: 14/372,672

(22) PCT Filed: Jan. 22, 2013
(86) PCT No.: PCT/JP2013/051210

§ 371 Date: Jul. 16, 2014

Related U.S. Application Data
(60) Provisional application No. 61/590,488, filed on Jan. 25, 2012.

(51) Int. Cl. C12N 15/82; C07K 14/415

(52) U.S. Cl. CPC C12N 15/8271; C07K 14/415
USPC 800/289; 800/298; 800/278

(57) ABSTRACT

[Problem] To impart an improved resistance against environmental stress to a plant body without inducing a delay in the growth or dwarfing of the plant body. [Solution] The present invention clarifies for the first time that Arabidopsis thaliana YC10 interacts with DREB2A. Also, the present invention clarifies for the first time that when a host plant is transformed with Arabidopsis thaliana NF-YC10 gene, the thus obtained transformant has an improved resistance to environmental stress.

### TECHNICAL FIELD

The present invention relates to a gene relating to the environmental stress resistance of plants and the application of recombinant technology utilizing this gene. In particular, it relates to the utilization of a gene to impart high temperature stress resistance to a plant.

### BACKGROUND ART

The demand for food is increasing with the rapid population growth of recent years, and there are also current statistics stating that over a billion people are facing hunger worldwide. In other words, the rate of increase in the food supply and the amount of arable land in the world does not meet the rate of increase in food demand. There also exist various problems with the stable production and supply of food crops, such as climate change, increasing demand for crops as an energy resource, and the like.

The production of crops having improved environmental stress resistance is considered as a means of resolving complex problems such as the above. Specifically, environmental stress is one of the most important factors affecting plant growth and is also a factor that significantly alters the yield in crop production. It is therefore possible that imparting environmental stress resistance to crops will make it possible to utilize currently unusable land as arable land and to suppress decreases in yield due to environmental stresses such as high temperature, drought, flooding, and low temperature during the growing season of the crops.

<DREB (DRE Binding Protein)>

Plants are known to be able to express various environmental stress resistance genes to avoid lethal injury under environmental stress conditions thereby protecting themselves. DRE (dehydration responsive element) is a sequence verified to exist by promoter analysis of RD29A, a water stress-inducible gene. The core sequence of DRE is said to consist of six bases A/GCCGAC (Non-patent Reference 1). DREB (DRE binding protein), a transcriptional activator, was also isolated by one-hybrid screening of yeast as a protein that binds to this sequence (Non-patent Reference 2). Two genes, DREB1A and DREB2A, were isolated in Non-patent Reference 2, but six DREB1 type genes and eight DREB2 type genes were subsequently confirmed in the genome of Arabidopsis thaliana (Non-patent Reference 3). All of these proteins have a highly conserved DNA binding domain (AP2/ERF domain), but it is reported that DREB1A, DREB1B, and DREB1C among the DREB1 type genes are induced mainly during low temperature stress while DREB2A and DREB2B among the DREB2A type genes are induced mainly during dehydration and salt stress (Non-patent Reference 2 and Non-patent Reference 4).

<DREB2A>

Since DREB2A has the ability to bind to DRE and is induced during dehydration and salt stress, it was assumed that it might function to improve the water stress resistance of plants. Nonetheless, no increase in the amount of mRNA of RD29A, an assumed target gene of the DREB2A protein, could be confirmed even when the DREB2A gene was overexpressed in a plant, and the resistance to dehydration stress also did not improve (Non-patent Reference 2). However, since transcription into mRNA of the DREB2A gene was confirmed even at that time, the possibility was suggested that the DREB2A protein is subject to post-translational regulation. It was subsequently clarified that when an NRD (negative regulatory domain) domain corresponding to amino acids 136-165 of the DREB2A protein is deleted, this NRD domain-deleted protein (called DREB2A CA: DREB2A constitutively active form) constitutively activates transcription of the target gene RD29A. The resistance to dehydration and salt stress also improved in plants that overexpressed DREB2A CA. In addition, with regard to the high transcriptional activation ability demonstrated by DREB2A CA, the DREB2A CA protein was suggested to be stabilized by deleting the NRD domain from DREB2A (Non-patent Reference 5).

Microarray analysis of plants that overexpress DREB2A CA protein clarified that not only expression of various dehydration and salt stress-inducible genes but also expression of high temperature stress-inducible genes rises in these plants (Non-patent References 5 and 6). In addition, Arabidopsis thaliana that overexpressed DREB2A CA protein was clarified to present improved resistance to not only dehydration and salt stress but also to high temperature stress (Non-patent Reference 6). Furthermore, OsDREB2B2 and GmDREB2A; 2 were also recently identified as homologous proteins of Arabidopsis thaliana DREB2A in Oryza sativa and Glycine max (Non-patent References 7 and 8).

Unfortunately, however, overexpression of DREB2A CA delays plant growth and causes dwarfing (Non-patent Reference 6).

<NF-Y>

NF-Y is a transcriptional control element known to date to be possessed by all eukaryotes. In NF-Y, NF-YA, NF-YB, and NF-YC are known to form a heteromeric trimer to regulate transcription (Non-patent references 9 and 10). Although extensive research has not yet been done in Arabidopsis thaliana and other such plants, there are also reports that the trimer acts on specific transcription factors and positively regulates the activity of the corresponding transcription factors (Non-patent References 11, 12, and 13).

Nonetheless, there have been no definitive reports to date on the function of NF-YC10, a type of Arabidopsis thaliana NF-YC protein.

### PRIOR ART REFERENCES

### Non-Patent References

Non-Patent Reference 1: Yamaguchi-Shinozaki and Shinozaki, Plant Cell, Vol. 6, pp. 251-264 (1994)

Non-Patent Reference 2: Liu et al., Plant Cell, Vol. 10, pp. 1391-1406 (1998)

Non-Patent Reference 3: Sakuma et al., Biochem. Biophys. Res. Commun., Vol. 290, pp. 998-1009 (2002)

Non-Patent Reference 4: Yamaguchi-Shinozaki and Shinozaki, Annu. Rev. Plant Biol., Vol. 57, pp. 781-803 (2006)

Non-Patent Reference 5: Sakuma et al., Plant Cell, Vol. 18, pp. 1292-1309 (2006)

Non-Patent Reference 6: Sakuma et al., Proc. Natl. Acad. Sci., Vol. 103, pp. 18822-18827 (2006)

Non-Patent Reference 7: Matsukura et al., Mol Genet Genomics, 2010, “Comprehensive analysis of rice DREB2-type genes that encode transcription factors involved in the expression of abiotic stress-responsive genes.”

Non-Patent Reference 8: Mizoi et al., Plant Physiol, 2013, “GmDREB2A; 2, a Canonical DEHYDRATION-RESPONSIVE ELEMENT-BINDING PROTEIN2-Type Transcription Factor in Soybean, Is Posttranslationally Regulated and Mediates Dehydration-Responsive Element-Dependent Gene Expression.”

Non-Patent Reference 9: Edwards et al., Plant Physiol., Vol. 117, pp. 1015-1022 (1998)

Non-Patent Reference 10: Mantovani, Gene, Vol. 239, pp. 15-27 (1999)

Non-Patent Reference 11: Yamamoto et al., Plant J., Vol. 58, pp. 843-856 (2009)

Non-Patent Reference 12: Liu et al., Plant Cell, Vol. 22, pp. 782-796 (2010)

Non-Patent Reference 13: Liu et al., Plant J., Vol. 67, pp. 763-773 (2011)

### SUMMARY OF THE INVENTION

### Problems to be Solved by the Invention

The purpose of the present invention is to impart resistance to environmental stress to a plant, in particular to impart resistance to high temperature stress to a plant. The plant to which this resistance to stress has been imparted should not exhibit delayed growth or dwarfing.

### Means Used to Solve the Above-Mentioned Problems

The present invention clarifies for the first time that Arabidopsis thaliana NF-YC10 interacts with DREB2A. The present invention also clarifies for the first time that, when a host plant is transformed by an Arabidopsis thaliana NF-YC10 gene, the resistance of the transformant to environmental stress improves. Surprisingly enough, the transformed plant exhibited equivalent growth to the original host plant, except for having improved resistance to environmental stress, without presenting growth delay or dwarfing.

Therefore, the first aspect of the present invention is:

<1> A transformed plant showing improved resistance to environmental stress that overexpresses a gene containing a nucleotide sequence selected from the group consisting of:

(1) a nucleotide sequence encoding a protein comprising an amino acid sequence shown by SEQ ID NO: 2, 4, 6, or 8;

(2) a nucleotide sequence encoding a protein having 60% or higher sequence homology to the amino acid sequence shown by SEQ ID NO: 2, 4, 6, or 8 and having an ability to bind to DREB2A protein; and

(3) a nucleotide sequence that hybridizes under stringent conditions with a nucleic acid comprising a nucleotide sequence complementary to a nucleotide sequence shown by SEQ ID NO: 1, 3, 5, 7, 14, 15, 16, or 17 and encodes a protein having the ability to bind to DREB2A protein.

Referring to a more specific embodiment, a sequence of a coding region of an Arabidopsis thaliana NF-YC10 gene (SEQ ID NO: 1), Oryza sativa NF-YC16 gene (SEQ ID NO: 3), Glycine max NF-YC22 (SEQ ID NO: 5), or Glycine max NF-YC23 (SEQ ID NO: 7) can be utilized as a nucleotide sequence of (1) above. Therefore, a preferred embodiment of the present invention is:

<2> The transformed plant according to <1> above, wherein the nucleotide sequence of (1) is a nucleotide sequence of a coding region of a nucleotide sequence shown by SEQ ID NO: 1, 3, 5, or 7.

In addition, regarding the “60% or higher sequence homology” of (2) above, the homology of an amino acid sequence of the present invention is defined as the positive percentage shown by the BLASTP algorithm that can be implemented by the internet site http://www.ncbi.n/m.nih.gov/egi-gin/BLAST by a search using the default parameters of the program (matrix=Blosum 62; gap cost: open=11, extend=1). Nevertheless, preferred examples are a sequence homology of 80% or higher, 90% or higher, and 95% or higher. Alternatively, one having a sequence identity percentage of 60%, 70%, 80%, 90%, or 95% or higher by this algorithm can also be given as an example of a homologous protein of the present invention. In other words, these homologous proteins may include homologous gene products of Arabidopsis thaliana NF-YC10, Oryza sativa NF-YC16, Glycine max NF-YC22, or Glycine max NY-YC23 and variants based on known gene recombination techniques, and it will be apparent to those skilled in the art by the disclosure of the present invention that these homologous gene products and variants can be utilized in the present invention as long as they retain the ability to bind substantially to DREB2A protein and interact therewith. Therefore, a preferred embodiment of the present invention includes:

<3> The transformed plant according to <1> above, wherein the sequence homology in (2) is 80% or higher.

The transformed plant of the present invention has been demonstrated to be able to exhibit improved resistance to high temperature stress in particular. Therefore, an especially preferred embodiment of the present invention is:

<4> The transformed plant according to any of <1> to <3> above, wherein the environmental stress is high temperature stress.

It will be readily understood that a form that accords with the use is preferred when utilizing the transformant of the present invention. In other words, in the case of a crop, the seedling form of the plant has an advantage in that it presents resistance to this stress even when exposed to high temperature stress when stored and distributed as a seedling. Of course, these seedlings can give plants that exhibit resistance to environmental stress over the entire period until the mature plant is utilized. Calluses of the transformant of the present invention can also be utilized to advantage for the purpose of research in the plant biotechnology field and the like. Therefore, other embodiments of the present invention are:

<5> The transformed plant according to any of <1> to <4> above, wherein the transformed plant is in a form of a seed;

<6> The transformed plant according to any of <1> to <4> above, wherein the transformed plant is in a form of a seedling; and

<7> The transformed plant according to any of <1> to <4> above, wherein the transformed plant is in a form of a callus.

The importance of monocotyledonous plants and dicotyledonous plants as food crops and horticultural crops goes without saying. Therefore, more preferred embodiments of the present invention are:

<8> The transformed plant according to any of <1> to <7> above, wherein the transformed plant is a dicotyledonous plant; and <9> The transformed plant according to any of <1> to <7> above, wherein the transformed plant is a monocotyledonous plant.

The present invention also intends a method for the production of these transformed plants. Those skilled in the art will appreciate that these transformed plants can be produced by inducing overexpression of the above genes in plant cells by introducing an exogenous gene into a host plant or by replacing or mutating an endogenous promoter that controls transcription of an endogenous gene. Therefore, the second aspect of the present invention is:

<10> A method for producing a transformed plant showing improved resistance to environmental stress, including:
i) a step for transforming a plant cell, wherein
   a) the plant cell is transfected by an expression vector containing a nucleotide sequence selected from the following group to cause overexpression of the gene containing the nucleotide sequence in the cell:
      (1) a nucleotide sequence encoding a protein comprising an amino acid sequence shown by SEQ ID NO: 2, 4, 6, or 8;
      (2) a nucleotide sequence encoding a protein having 60% or higher sequence homology to the amino acid sequence shown by SEQ ID NO: 2, 4, 6, or 8 and having an ability to bind to DREB2A protein; and
      (3) a nucleotide sequence that hybridizes under stringent conditions with a nucleic acid comprising a nucleotide sequence complementary to a nucleotide sequence shown by SEQ ID NO: 1, 3, 5, 7, 14, 15, 16, or 17 and encodes a protein having the ability to bind to DREB2A protein; or
   b) a control region of an endogenous gene containing a nucleotide sequence selected from the group of a) (1)-(3) above is replaced by an exogenous control element in the plant cell to cause overexpression of the gene in the cell; and
ii) a step for causing a growth of a transformed plant cell obtained in step i) above under conditions suited to regeneration of a plant from the cell to obtain a transformed plant.

Embodiments described for the first aspect of the present invention also apply to the second aspect of the present invention. These embodiments include:

<11> The method for producing a transformed plant according to <10> above, wherein the nucleotide sequence of (1) is a nucleotide sequence of a coding region of a nucleotide sequence shown by SEQ ID NO: 1, 3, 5, or 7;

<12> The method for producing a transformed plant according to <10> above, wherein the sequence homology in (2) is 80% or higher;

<13> The method for producing a transformed plant according to any of <10> to <12> above, wherein the environmental stress is high temperature stress;

<14> The method for producing a transformed plant according to any of <10> to <13> above, wherein the transformed plant is in a form of a seed;

<15> The method for producing a transformed plant according to any of <10> to <13> above, wherein the transformed plant is in a form of a seedling;

<16> The method for producing a transformed plant according to any of <10> to <13> above, wherein the transformed plant is in a form of a callus;

<17> The method for producing a transformed plant according to any of <10> to <16> above, wherein the transformed plant is a dicotyledonous plant; and

<18> The method for producing a transformed plant according to any of <10> to <16> above, wherein the transformed plant is a monocotyledonous plant.

The present invention also intends a method for improving the resistance of a plant to environmental stress by the advantages of the present invention described above. Therefore, the third aspect of the present invention and related embodiments include:

<19> A method for improving resistance of a plant to environmental stress, including:
a) transfecting a plant cell by an expression vector containing a nucleotide sequence selected from the following group to cause overexpression of a gene containing the nucleotide sequence in the cell:
   (1) a nucleotide sequence encoding a protein comprising an amino acid sequence shown by SEQ ID NO: 2, 4, 6, or 8;
   (2) a nucleotide sequence encoding a protein having 60% or higher sequence homology to the amino acid sequence shown by SEQ ID NO: 2, 4, 6, or 8 and having an ability to bind to DREB2A protein; and
   (3) a nucleotide sequence that hybridizes under stringent conditions with a nucleic acid comprising a nucleotide sequence complementary to a nucleotide sequence shown by SEQ ID NO: 1, 3, 5, 7, 14, 15, 16, or 17 and encodes a protein having the ability to bind to DREB2A protein; or
b) replacing a control region of an endogenous gene containing a nucleotide sequence selected from the group of a) (1)-(3) above by an exogenous control element in the plant cell to cause overexpression of the gene in the cell;

<20> The method according to <19> above, wherein the nucleotide sequence of (1) is a nucleotide sequence of a coding region of a nucleotide sequence shown by SEQ ID NO: 1, 3, 5, or 7;

<21> The method according to <19> above, wherein the sequence homology in (2) is 80% or higher;

<22> The method according to any of <19> to <21> above, wherein the environmental stress is high temperature stress;

<23> The method according to any of <19> to <22> above, wherein the transformed plant is in a form of a seed;

<24> The method according to any of <19> to <22> above, wherein the transformed plant is in a form of a seedling;

<25> The method according to any of <19> to <22> above, wherein the transformed plant is in a form of a callus;

<26> The method according to any of <19> to <25> above, wherein the plant is a dicotyledonous plant; and

<27> The method according to any of <19> to <25> above, wherein the plant is a monocotyledonous plant.

As it were, another aspect of the present invention is:

<28> A gene to be used to improve resistance of a plant to environmental stress, containing a nucleotide sequence selected from a group consisting of:

(1) a nucleotide sequence encoding a protein comprising an amino acid sequence shown by SEQ ID NO: 2, 4, 6, or 8;

(2) a nucleotide sequence encoding a protein having 60% or higher sequence homology to the amino acid sequence shown by SEQ ID NO: 2, 4, 6, or 8 and having an ability to bind to DREB2A protein; and

(3) a nucleotide sequence that hybridizes under stringent conditions with a nucleic acid comprising a nucleotide sequence complementary to a nucleotide sequence shown by SEQ ID NO: 1, 3, 5, 7, 14, 15, 16, or 17 and encodes a protein having the ability to bind to DREB2A protein.

### Advantages of the Invention

According to the present invention, the resistance of a plant to environmental stress, in particular the resistance of a plant to high temperature stress can be improved.

### BRIEF DESCRIPTION OF THE DRAWINGS

[FIG. 1] FIG. 1 shows the DNA sequence of an Arabidopsis thaliana NF-YC10 gene (SEQ ID NO: 1). Bases 18-638 are the coding region.

[FIG. 2] FIG. 2 shows the DNA sequence of an Oryza sativa NF-YC16 gene (SEQ ID NO: 3). Bases 111-1022 are the coding region.

[FIG. 3] FIG. 3 shows the DNA sequence of a Glycine max NF-YC22 gene (SEQ ID NO: 5). Bases 98-658 are the coding region.

[FIG. 4] FIG. 4 shows the DNA sequence of a Glycine max NF-YC23 gene (SEQ ID NO: 7). Bases 93-650 are the coding region.

[FIG. 5] FIG. 5 shows the results of interaction analysis of DREB2A protein and Arabidopsis thaliana NF-YC10 protein by a yeast two-hybrid system. The results show that yeast with both “NF-YC10 Full” and “DREB2A (1-205)+BD” introduced exhibits the ability to grow even on SD/-Leu/-Trp/-His/-Ade/3-AT (QDO) agar medium.

[FIG. 6] FIG. 6 shows the results of interaction analysis (BiFC study) of DREB2A protein and Arabidopsis thaliana NF-YC10 protein by transient expression using Arabidopsis thaliana protoplasts. Only protoplasts with “NF-YC10 Full+DREB2A Full” introduced generate a YFP fluorescence signal. “bZIP63+bZIP63” is a positive control already confirmed to interact. CFP is introduced simultaneously to confirm proper gene introduction.

[FIG. 7] FIG. 7 shows overexpression of the gene in Arabidopsis thaliana with an Arabidopsis thaliana NF-YC10 gene introduced. 35S:NF-YC10-a, 35S:NF-YC10-b, and 35S:NF-YC10-c are three independent lines having a high expression level of the Arabidopsis thaliana NF-YC10 introduced. VC is a control transformed by only an empty vector. The upper row is the results of northern analysis of NF-YC10 by probe DNA. The lower row is the ethidium bromide-stained control.

[FIG. 8] FIG. 8A shows the growth state of the rosette leaves of three lines of transformed plants of the present invention (35S:NF-YC10-a, 35S:NF-YC10-b, and 35S:NF-YC10-c) after being grown for 16 days from sowing on 1% sucrose-containing GMK medium under stress-free conditions. FIG. 8B shows the state after the three lines of transformed plants were transplanted to pots and grown for another two weeks after being grown for two weeks from sowing on 1% sucrose-containing GMK medium under stress-free conditions. The vector control is a plant transformed by an empty vector.

[FIG. 9] FIG. 9A is a graph showing the results obtained by measuring the results of FIG. 8A as the maximum radius of the rosette leaves. FIG. 9B is a graph showing the results obtained by measuring the results of FIG. 8B as the inflorescence length.

[FIG. 10] FIG. 10 shows the results of expression analysis of the target gene of DREB2A in NF-YC10-overexpressing Arabidopsis thaliana. In the figure, three lines of transformed plants expressing high levels of Arabidopsis thaliana NF-YC10 of the present invention (35S:NF-YC10-a, 35S:NF-YC10-b, and 35S:NF-YC10-c) are compared with a vector control (negative control) transformed by an empty vector.

[FIG. 11] FIG. 11 shows the results of microarray analysis after high temperature stress using two separate lines of transformants having significant changes in the expression levels of HsfA3 and At1g75860, which are downstream genes of DREB2A, in NF-YC10-overexpressing Arabidopsis thaliana. Fifteen genes that induced twice or more the expression level of the vector control plants were discovered.

[FIG. 12] FIG. 12 shows the results of a high temperature stress resistance test of NF-YC10-overexpressing Arabidopsis thaliana of the present invention (35S:NF-YC10-a, 35S:NF-YC10-b, and 35S:NF-YC10-c). Comparison was made with a vector control (negative control) transformed by an empty vector. The numbers in denominator and numerator in parentheses in the figure represent the number of plants used in the test and the number of plants that survived, respectively. The survival rate (%) is also shown in the figure.

[FIG. 13] FIG. 13 shows the homologous genes corresponding to the dicotyledonous Arabidopsis thaliana NF-YC10 and the designations assigned by the present inventors in Glycine max, the monocotyledonous Oryza sativa, the moss Physcomitrella patens, and the blue-green algae Chlamydomonas and Volvox.

[FIG. 14] FIG. 14 shows the relationships of Arabidopsis thaliana NF-YC10 homologous genes and human, mouse, and yeast NF-YC family genes by a phylogenetic tree. The black dots in the figure show a bootstrap value of 50 or higher.

[FIG. 15] FIG. 15 shows a comparison of the amino acid sequences of the conserved regions of Arabidopsis thaliana (NF-YC10: SEQ ID NO: 24) and the most closely related homologous genes to Arabidopsis thaliana NF-YC10 in Oryza sativa (OsNF-YC16: SEQ ID NO: 25), Glycine max (GmNF-YC22: SEQ ID NO: 26 and GmNF-YC23: SEQ ID NO: 27), and Physcomitrella patens (PpNF-YC11: SEQ ID NO: 28). Amino acid residues that are highly similar in all sequences are shown in white. Amino acid residues that are highly similar in three or more sequences are shown in bold.

[FIG. 16] FIG. 16 shows the results of interaction analysis of (Glycine max) GmDREB2A; 2 protein and (Oryza sativa) OsDREB2B2 protein with Arabidopsis thaliana NF-YC10 protein by a yeast two-hybrid system. The results show that yeast having both “NF-YC10 full+AD” and “GmDREB2A; 2 (1-137 a.a)+BD” introduced and yeast having both “NF-YC10 full+AD” and “OsDREB2B2 (1-146 a.a)+BD” introduced exhibited the ability to grow even on SD/-Leu/-Trp/-His/-Ade/3-AT (QDO) agar medium.

[FIG. 17] FIG. 17 shows the results of interaction analysis (BiFC study) of (Glycine max) GmDREB2A; 2 protein and (Oryza sativa) OsDREB2B2 protein with Arabidopsis thaliana NF-YC10 protein by transient expression using Arabidopsis thaliana protoplasts. Only the protoplast with “NF-YC10 full+GmDREB2A; 2 full” introduced and the protoplast with “NF-YC10 full+OsDREB2B2 full” introduced generate a YFP fluorescence signal. “bZIP63+bZIP63” is a positive control already confirmed to interact. CFP is introduced simultaneously to confirm proper gene introduction.

### BEST MODE FOR CARRYING OUT THE INVENTION

### Gene

The problem of the present invention is solved by inducing functional overexpression of an Arabidopsis thaliana NF-YC10 gene or a homologous gene thereof within the cells of a plant that is to have improved resistance to environmental stress. More specifically, the product of the functionally expressed Arabidopsis thaliana NF-YC10 gene or homologous gene thereof presents the ability to bind to DREB2A protein, and improved resistance to environmental stress is imparted to the transformed plant as a result of such functional expression.

An Arabidopsis thaliana NF-YC10 gene (SEQ ID NO: 1) encodes Arabidopsis thaliana NF-YC10 protein containing the following amino acid sequence.

[Chemical Formula 1]
(SEQ ID NO: 2)
Met Val Ser Ser Lys Lys Pro Lys Glu Lys Lys Ala
Arg Ser Asp Val Val Val Asn Lys Ala Ser Gly Arg
Ser Lys Arg Ser Ser Gly Ser Arg Thr Lys Lys Thr
Ser Asn Lys Val Asn Ile Val Lys Lys Lys Pro Glu
Ile Tyr Glu Ile Ser Glu Ser Ser Ser Ser Asp Ser
Val Glu Glu Ala Ile Arg Gly Asp Glu Ala Lys Lys
Ser Asn Gly Val Val Ser Lys Arg Gly Asn Gly Lys
Ser Val Gly Ile Pro Thr Lys Thr Ser Lys Asn Arg
Glu Glu Asp Asp Gly Gly Ala Glu Asp Ala Lys Ile
Lys Phe Pro Met Asn Arg Ile Arg Arg Ile Met Arg
Ser Asp Asn Ser Ala Pro Gln Ile Met Gln Asp Ala
Val Phe Leu Val Asn Lys Ala Thr Glu Met Phe Ile
Glu Arg Phe Ser Glu Glu Ala Tyr Asp Ser Ser Val
Lys Asp Lys Lys Lys Phe Ile His Tyr Lys His Leu
Ser Ser Val Val Ser Asn Asp Gln Arg Tyr Glu Phe
Leu Ala Asp Ser Val Pro Glu Lys Leu Lys Ala Glu
Ala Ala Leu Glu Glu Trp Glu Arg Gly Met Thr Asp
Ala Gly

As in the examples below, it is apparent that Arabidopsis thaliana NF-YC10 protein has the ability to substantially bind to and interact with DREB2A protein, thereby elevating expression of DREB2A downstream genes, especially high temperature-inducible genes.

An Oryza sativa NF-YC16 gene (SEQ ID NO: 3) is also shown by the results of phylogenetic tree analysis described below to be a homologous gene of Arabidopsis thaliana NF-YC10. Therefore, inducing overexpression of the Oryza sativa NF-YC16 gene within plant cells can impart improved environmental stress resistance to the plant. The Oryza sativa NF-YC16 gene encodes Oryza sativa NF-YC16 protein containing the following amino acid sequence.

[Chemical Formula 2]
(SEQ ID NO: 4)
Met Ala Gly Lys Lys Lys Ala Leu Thr Asn Pro Ala
Ser Pro Ser Ala Ser Ala Ser Ala Ser Thr Pro Lys
Lys Ser Thr Ala Thr Ser Lys Asp Arg Ser Thr Pro
Lys Pro Arg Lys Asn Pro Asn Pro Lys Glu Glu Ala
Pro Pro Pro Pro Pro Ala Asn Asn Lys Arg Leu Asn
Pro Gln Gly Gly Ser Asn Arg Lys Lys Lys Ala Asp
Ala Gly Thr Pro Ser Lys Lys Pro Lys Arg Gln Pro
Pro Glu Pro Lys Pro Arg Lys His Lys Gly Ala Lys
Ser Glu Lys Pro His Arg Val Ser Gly Glu Gly Glu
Lys Pro Thr Pro Thr Lys Lys Lys Lys Lys Lys Glu
Ser Ser Lys Glu Pro Lys Arg Glu Lys Gln Gln Ala
Ser Ala Pro Met Ser Thr Pro Ser Lys Lys Asn Lys
Glu Ala Lys Arg Asp Thr Gly Gly Ala Gly Lys Pro
Thr Pro Thr Lys Arg Lys Leu Gly Asp Val Asp Pro
Pro Gln Glu Arg Pro Ser Gly Glu Gly Gln Ala Ser
Ser Pro Thr Pro Ala Lys Lys Arg Lys Asp Lys Ala
Ala Ala Ala Glu Ala Val Ala Asp His Gly Ala Gly
Ser Phe Pro Met Ala Arg Val Arg Gln Ile Met Arg
Ala Glu Asp Ala Thr Ile Arg Pro Ser Asn Glu Ala
Val Phe Leu Ile Asn Lys Ala Thr Glu Ile Phe Leu
Lys Arg Phe Ala Asp Asp Ala Tyr Arg Asn Ala Leu
Lys Asp Arg Lys Lys Ser Ile Val Tyr Asp Asn Leu
Ser Thr Ala Val Cys Asn Gln Lys Arg Tyr Lys Phe
Leu Ser Asp Phe Val Pro Gln Lys Val Thr Ala Glu
Asp Ala Leu Lys Ala Pro Val Ser Ser Gln Val Asn
Gln Pro Gln

A Glycine max NF-YC22 gene (SEQ ID NO: 5) and Glycine max NF-YC23 gene (SEQ ID NO: 7) are also shown by the results of phylogenetic tree analysis described below to be homologous genes of Arabidopsis thaliana NF-YC10. Therefore, inducing overexpression of the Glycine max NF-YC22 gene and Glycine max NF-YC23 gene within plant cells can impart improved environmental stress resistance to the plant. Furthermore, it is thought from analysis of the genomic sequence that the Glycine max chromosome is diploid originating from tetraploid, and many genes are present in duplication. Given their high homology, Glycine max NF-YC22 protein and Glycine max NF-YC23 protein are presumed to be proteins present in duplication due to polyploidy. It is therefore reasonably understandable that both will exhibit bioactivity similar to the Arabidopsis thaliana NF-YC10 protein. The Glycine max NF-YC22 gene and Glycine max NF-YC23 gene encode Glycine max NF-YC22 protein and Glycine max NF-YC23 protein containing the following amino acid sequences, respectively.

[Chemical Formula 3]
< G m N F - Y C 2 2 >
(SEQ ID NO: 6)
Met Ala Ser Ser Asn Thr Pro Lys Pro Glu Asn Lys
Lys Ser Thr Lys Lys Ser Glu Ile Ser Lys Ala Glu
Lys Lys Lys Thr Lys Asn Ala Glu Ile Pro Lys Thr
Asp Gly Lys Thr Lys Lys Asn Lys Glu Ile Ser Gln
Glu Glu Asn Lys Lys Lys Ile Lys Lys Ala Lys Leu
Ser Asn Gly Thr Ser Lys Gln Arg Asp Glu Gly Ser
Lys Lys Gly Val Ala Ala Glu Gly Asn Gly Glu Glu
Ala Lys Met Asn Val Phe Pro Met Asn Arg Ile Arg
Thr Met Ile Lys Gly Glu Asp Pro Glu Met Arg Val
Ser Gln Glu Ala Leu Phe Ala Ile Asn Asn Thr Val
Glu Lys Phe Leu Glu Gln Phe Thr Gln Asp Ala Tyr
Ala Phe Cys Ala Gln Asp Arg Lys Lys Cys Leu Ser
Tyr Asp His Leu Ala His Val Val Ser Lys Gln Arg
Arg Tyr Asp Phe Leu Ser Asp Phe Val Pro Glu Arg
Val Lys Ala Glu Asp Ala Leu Arg Glu Arg Ser Ala
Ala Gly Lys Gly Gly Ser
< G m N F - Y C 2 3 >
(SEQ ID NO: 8)
Met Thr Ser Ser Asn Ser Pro Lys Pro Glu Lys Lys
Glu Lys Lys Lys Asn Ala Glu Ile Pro Lys Ile Glu
Lys Lys Lys Thr Lys Ser Ala Glu Ile Pro Leu Thr
Asp Gly Lys Thr Lys Arg Asp Arg Glu Ile Ala Lys
Glu Glu Asn Lys Lys Lys Thr Lys Lys Pro Lys Leu
Ser Asn Gly Thr Ser Lys Gln Arg Asp Glu Gly Ser
Lys Lys Gly Val Ala Glu Gly Lys Gly Glu Glu Gly
Lys Met Asn Val Phe Pro Met Asn Arg Ile Arg Thr
Met Ile Lys Gly Glu Asp Pro Asp Met Arg Val Ser
Gln Glu Ala Leu Leu Ala Ile Asn Asn Ala Val Glu
Lys Phe Leu Glu Gln Phe Ser Gln Glu Ala Tyr Ala
Phe Cys Val Arg Asp Arg Lys Lys Cys Leu Ser Tyr
Asp His Leu Ala His Val Val Ser Lys Gln Arg Arg
Tyr Asp Phe Leu Ser Asp Phe Val Pro Glu Arg Val
Lys Ala Glu Asp Ala Leu Arg Glu Arg Ser Ala Ala
Gly Thr Gly Gly His

A Physcomitrella patens NF-YC11 gene can also be given as an example of a homologous gene of Arabidopsis thaliana NF-YC10 (refer to the phylogenetic tree analysis described below). The Oryza sativa NF-YC16 protein and Physcomitrella patens NF-YC11 protein show positive percentages of approximately 81% or higher and approximately 68% or higher, respectively, to Arabidopsis thaliana NF-YC10 protein as amino acid sequence homology based on the BLASTP algorithm mentioned above. The Glycine max NF-YC22 protein and Glycine max NF-YC23 protein also show positive percentages of approximately 63% or higher and approximately 74% or higher, respectively, to Arabidopsis thaliana NF-YC10 protein. Those skilled in the art will consequently appreciate that genes encoding homologous proteins showing positive percentages of 60%, 70%, 80%, 90%, or 95% or higher to amino acid sequences of SEQ ID NO: 2, SEQ ID NO: 4, SEQ ID NO: 6, or SEQ ID NO: 8, for example, can be utilized in the present invention. Modified proteins of Arabidopsis thaliana NF-YC10, Oryza sativa NF-YC16, Glycine max NF-YC22, and Glycine max NF-YC23 are considered to be homologous proteins of the present invention as long as they have substantially similar activity to the protein of the wild type even if the structure of one of the modified protein molecules is not present in the wild type protein molecule or the two amino acid sequences are not exactly the same. For example, it is possible that replacing leucine by valine, lysine by arginine, and glutamine by asparagine will not change the function of a polypeptide. Therefore, proteins having amino acid sequences containing deletion, insertion, addition, and/or replacement of several amino acids relative to the amino acid sequences of SEQ ID NOS: 2, 4, 6, or 8, for example, can be utilized in the present invention as long as they retain the ability to interact by binding substantially with DREB2A protein. Those skilled in the art should also understand that the nucleotide sequences encoding these proteins can also be utilized in the present invention.

Furthermore, the interactivity of DREB2A and NF-YC10 homologous proteins can be confirmed by either or both a yeast two-hybrid system or a BiFC (bimolecular fluorescence complementation) study using an Arabidopsis thaliana protoplast system. Specifically, in a typical example of this two-hybrid system, cDNA (bases 1 to 615) encoding a region excluding the transcriptional activation domain on the C terminal side of DREB2A is ligated to a pGBKT7 plasmid (manufactured by Clontech), and full-length cDNA of NF-YC10 homologous protein is ligated to a pGADT7 plasmid (manufactured by Clontech). These plasmids are transformed into AH109 yeast, and DREB2A and the NF-YC10 homologous protein are judged to interact if growth of the yeast is seen on selection medium. In this case, plasmid DNA can be introduced into the yeast by the method of Ito et al. [Ito et al., J. Bacteriol., Vol. 153, pp. 163-168 (1983)], and the other study conditions may accord with the Matchmaker System manufactured by Clontech. In a typical example of a BiFC study, full-length cDNA of DREB2A is ligated to a pBI221 plasmid (manufactured by Clontech) containing a CaMV35S promoter and cDNA (bases 466 to 717) encoding the C terminal side of YFP. Similarly, full-length cDNA of an NF-YC10 homologous protein is ligated to a pBI221 plasmid containing a CaMV35S promoter and cDNA (bases 1 to 465) encoding the N terminal side of YFP. After then introducing these two plasmids into Arabidopsis thaliana protoplasts, MG132, a proteasome inhibitor, is added to improve the stability of DREB2A. If YFP fluorescence can be observed thereafter, DREB2A and the NF-YC10 homologous protein are judged to interact within the protoplasts. In this case, protoplast preparation and introduction of plasmid DNA into the protoplasts can be carried out by the method of Yoo et al. [Yoo et al., Nat. Protoc., Vol. 2, pp. 1565-1572 (2004)].

As another explanation of genes that can be utilized in the present invention, conserved regions identified by alignment of the amino acid sequences of Arabidopsis thaliana NF-YC10 protein, Oryza sativa NF-YC16 protein, Glycine max NF-YC22 and NF-YC23 protein, and Physcomitrella patens NF-YC11 protein are shown in FIG. 15. The following relatively long common amino acid sequences were discovered in these conserved regions.

[Chemical Formula 4]
N F - Y C 1 0
(SEQ ID NO: 9)
R Y E F L A D S V P E K L K A E A A L;
O s N F - Y C 1 6:
(SEQ ID NO: 10)
R Y K F L S D F V P Q K V T A E D A L;
G m N F - Y C 2 2:
(SEQ ID NO: 11)
R Y D F L S D F V P E R V K A E D A L;
G m N F - Y C 2 3:
(SEQ ID NO: 12)
R Y D F L S D F V P E R V K A E D A L;
P p N F - Y C 1 1:
(SEQ ID NO: 13)
R L E F L S D I V P V R I P A A A A L

Therefore, those skilled in the art will also appreciate that genes overexpressed for purposes of the present invention can be identified as nucleic acids that hybridize under stringent conditions to a nucleotide complementary to a nucleotide encoding an above-mentioned common amino acid sequence and can be obtained easily by confirming that their expression products bind to DREB2A protein. For example, partial sequences encoding the above common amino acid sequences of Arabidopsis thaliana NF-YC10 protein and Oryza sativa NF-YC16 protein contain nucleotide sequences of the following SEQ ID NO: 14 and SEQ ID NO: 15, respectively; therefore, nucleic acids that hybridize under stringent conditions to either or both nucleic acids containing sequences complementary to these nucleotide sequences can be utilized in the present invention. Alternatively, partial sequences encoding amino acid sequences of the above conserved regions in Glycine max NF-YC22 protein and Glycine max NF-YC23 contain nucleotide sequences of the following SEQ ID NO: 16 and SEQ ID NO: 17, respectively; therefore, nucleic acids that hybridize under stringent conditions to either or both nucleic acids containing sequences complementary to these nucleotide sequences can be utilized in the present invention. Furthermore, stringent conditions in this specification are described as hybridization at 42° C. in solution containing denatured salmon sperm DNA, 6×SSC solution and 5× Denhart solution and washing at 68° C. in aqueous solution containing 0.1% SDS and 1×SSC.

[Chemical Formula 5]
N F - Y C 1 0:
(SEQ ID NO: 14)
A G A T A C G A G T T C C T T G C A G A T A G T G
T T C C C G A G A A A C T T A A A G C A G A G G C
C G C G T T G;
O s N F - Y C 1 6:
(SEQ ID NO: 15)
A G A T A C A A G T T T C T C T C A G A T T T T G
T T C C A C A G A A A G T T A C A G C T G A A G A
T G C T T T G;
G m N F - Y C 2 2:
(SEQ ID NO: 16)
A G A T A T G A C T T T C T C T C T G A T T T T G
T T C C T G A G A G A G T A A A A G C T G A G G A
T G C A T T A;
G m N F - Y C 2 3:
(SEQ ID NO: 17)
A G A T A T G A C T T T C T C T C T G A T T T T G
T T C C T G A G A G A G T G A A A G C T G A G G A
T G C A T T A

Furthermore, homologous genes of Arabidopsis thaliana NF-YC10 that can be utilized for purposes of the present invention may have all mutations that can occur in nature and artificially introduced mutations and modifications as long as their expression product is capable of interaction by substantially binding to DREB2A protein. For example, the presence of excess codons (redundancy) is known in various codons that encode a specific amino acid. Alternate codons that are finally translated into the same amino acid may therefore also be utilized in the present invention. In other words, since the genetic code degenerates, multiple codons can be used to encode a certain specific amino acid, and the amino acid sequence can therefore be encoded by any one set of similar DNA oligonucleotidesto. While only one member of that set is identical to the native genetic sequence (for example, SEQ ID NO: 1, SEQ ID NO: 3, SEQ ID NO: 5, or SEQ ID NO: 7), even DNA oligonucleotides with mismatch can hybridize to the native sequence under stringent conditions, and DNA that encodes the native sequence can be identified and isolated. Such genes can also be utilized in the present invention. In particular, since virtually all organisms are known to use subsets of specific codons (optimal codons) preferentially (Gene, Vol. 105, pp. 61-72, 1991, and the like), “codon optimization” in accordance with the host can also be useful in the present invention.

### Expression Vector

The above genes are overexpressed within transformed plants in the present invention. Such transformation is typically achieved by transfecting a plant cell by an expression vector containing an exogenous above gene. Furthermore, in this specification, the term “exogenous” is used to mean that a gene or nucleotide sequence based on the present invention is introduced into a host in a case in which the host plant prior to transformation does not have the gene to be introduced by the present invention, a case in which it substantially does not express the protein encoded by this gene, and a case in which the amino acid sequence of this protein is encoded by a different gene, but endogenous protein activity comparable to that after transformation is not expressed. In this specification as well, “expression vector” means a nucleotide containing a nucleotide sequence that regulates transcription and translation functionally linked to the nucleic acid to be expressed or the gene to be expressed. Typically, an expression vector of the present invention contains a promoter sequence 5′ upstream from the coding sequence, a terminator sequence 3′ downstream, and sometimes also a normal regulatory element in a functionally linked state. In such cases, the nucleic acid to be expressed or the gene to be expressed is introduced expressibly into the host plant cell.

Specifically, a (recombination) expression vector for introduction into a plant that can preferably be utilized in the present invention can be obtained by cleaving DNA containing a gene of the present invention by suitable restriction enzymes, linking suitable linkers as needed, and inserting into a cloning vector for plant cells. pBE2113Not, pBI2113Not, pBI2113, pBI101, pBI121, pGA482, pGAH, pBIG, pGreen, and other such plasmid binary vectors and pLGV23Neo, pNCAT, pMON200, and other such plasmid intermediate vectors can be used as cloning vectors. When a plasmid binary vector is used, the target gene is inserted between the border sequences (LB, RB) of the binary vector, and this recombinant vector is amplified in E. coli. Next, the amplified recombinant vector is introduced into Agrobacterium tumefaciens C58, LBA4404, EHA101, C58C1RifR, EHA105, or the like by a freeze-thaw process, electroporation, or the like, and this agrobacterium is used to transform the plant.

As was mentioned above, a promoter, terminator, and the like for each plant must be positioned in front of and behind the structural gene to cause an exogenous gene and the like to be expressed within the plant. Examples of promoters that can be utilized in the present invention include promoters of 35S transcripts from cauliflower mosaic virus (CaMV) [Jefferson et al., The EMBO J., Vol. 6, pp. 3901-3907 (1987)], corn ubiquitin [Christensen et al., Plant Mol., Vol. 18, pp. 675-689 (1992)], nopaline synthase (NOS) gene, octopine (OCT) synthase gene, and the like. Examples of terminator sequences include a terminator from a nopaline synthase gene from cauliflower mosaic virus and the like. However, they are not limited to these as long as they are promoters and terminators known to function within plants.

Intron sequences that function to enhance gene expression, for example, a corn alcohol dehydrogenase (Adh1) intron [Genes & Development, Vol. 1, pp. 1183-1200 (1987)], can also be introduced between the promoter sequence and gene of the present invention as needed. Moreover, effective selection marker genes are preferably used in combination with the gene of the present invention to efficiently select the target transformed cells. One or more genes selected from a kanamycin resistance gene (NPTII), hygromycin phosphotransferase (htp) gene to impart resistance to the antibiotic hygromycin to a plant, phosphinothricin acetyltransferase (bar) gene to impart resistance to bialaphos, and the like can be used as the selection markers used in this case. The gene of the present invention and the selection marker gene may be incorporated together into a single vector or two types of recombinant DNA incorporated into individual vectors may be used.

### Transformation

The host of the transformant of the present invention may be any of cultured plant cells, calluses, the whole plant of cultivated plants, plant organs (for example, leaves, petals, stems, roots, rhizomes, seeds, and the like), or plant tissues (for example, epidermis, phloem, parenchyma, xylem, vascular bundle, and the like). The plant species is not restricted; Glycine max and other such dicotyledonous plants, Oryza sativa, Zea mays, Triticum aestivum, and other such monocotyledonous plants can also be used. When the plant being transformed is a dicotyledonous plant, it is preferable to introduce a gene of the present invention from a dicotyledonous plant such as Arabidopsis thaliana or the like. When the plant being transformed is a monocotyledonous plant, it is preferable to introduce a gene of the present invention from a monocotyledonous plant such as Oryza sativa or the like. When cultured plant cells, whole plant, plant organs, or plant tissues serve as the host, the plant host can be transformed by introducing a vector containing DNA that encodes the protein of the present invention by the agrobacterium infection method, particle gun method, polyethylene glycol method, or the like into the plant sections collected. Alternatively, a transformed plant can also be produced by introduction into protoplasts by electroporation.

For example, when introducing a gene into Arabidopsis thaliana by the agrobacterium infection method, a step that transfects agrobacterium carrying a plasmid containing the target gene into the plant is essential. This can be done by a modification of the floral dip method [Clough et al., Plant J., Vol. 16, pp. 735-743 (1998)]. More specifically, buds of Arabidopsis thaliana grown in soil containing vermiculite, pearlite, and the like are dipped directly in bacterial solution obtained by suspending agrobacterium having a plasmid containing the gene of the present invention in infiltration medium (0.5×MS salt, 5% (w/v) sucrose, 10 μg/L benzyladenine, 0.05% (v/v) Silwet L-77), and humidity is maintained by covering the pot by plastic wrap. The next day, the wrap is removed; the plants are allowed to grow as they are, and the seeds are harvested. Next, the seeds are sown on GM agar medium with a suitable antibiotic added to select individuals having the target gene from among the seeds. The Arabidopsis thaliana grown by this medium are transferred to pots, and seeds of transformed plants having the gene of the present invention introduced can be obtained by causing the plants to grow. In other words, plants of the present invention can be reproduced suitably from cells and the like transfected by the gene of the present invention by causing a growth under such conditions.

The DNA may be extracted by the usual method from these cells or tissues, and the gene introduced may be detected using known PCR or southern methods to confirm that the target gene is incorporated in transformed plants having the gene of the present invention introduced or subsequent generations thereof. Furthermore, the transgene is generally introduced into the genome of the host plant, but a phenomenon called position effect is known whereby expression of the transgene differs depending on differences in the location at which it is introduced. Transformants that express the transgene more strongly can therefore be selected by detection by the northern method using DNA fragments of the transgene as a probe.

An example of another method of the present invention is to significantly activate expression of an endogenous gene of the above host plant. Specifically, a regulatory region, for example, a promoter, of said endogenous gene may be replaced by an exogenous control element such as the above promoters of 35S transcripts from cauliflower mosaic virus (CaMV), corn ubiquitin, nopaline synthase (NOS) gene, octopine (OCT) synthesis gene, and the like. Transformed plants having significantly activated expression of an endogenous gene by such a method can be obtained by selecting individuals that more strongly express the gene by detection by the northern method using DNA fragments of said endogenous gene as a probe. In other words, the expression level and expression site of the gene in transformed plants having the gene of the present invention introduced can be analyzed by extracting RNA by the usual method from their cells or tissues and detecting the mRNA of the gene introduced using known RT-PCR or northern methods. Alternatively, as another method, the gene product of the present invention can also be analyzed directly by western analysis using an antibody to the gene product or the like.

### Characterization of Transformed Plants

As was mentioned above, the gene of the present invention acts as a transcriptional control element. The gene group believed to have a changed transcription level in transformed plants having the gene of the present invention introduced can be identified by the northern method. For example, environmental stress is applied for a certain length of time (for example, 30 minutes to 24 hours) to plants grown in GM agar medium or the like. Examples of environmental stress include drying, high temperature stress, and the like involving DREB2A. For example, drought stress can be applied by removing the plant from the GM agar medium and leaving it on filter paper for from 30 minutes to 24 hours. High temperature stress can be applied by leaving a plant grown on GM agar medium for from 30 minutes to 24 hours at 37° C. Total RNA is prepared from unstressed control plants and plants to which environmental stress has been applied. Electrophoresis is then carried out, and changes in the expression pattern can be analyzed by the northern method using DNA fragments of the gene confirmed to be expressed as a probe.

### Evaluation of Transformed Plant Tesistance to Environmental Stress

The resistance of transformed plants having the gene of the present invention introduced to environmental stress can be evaluated by applying various types of environmental stress to, for example, plants transplanted to pots containing soil containing vermiculite, pearlite, and the like after being grown for a certain length of time (for example, 2-3 weeks) on GM again medium or plants grown on filter paper soaked with GM liquid medium and studying their survival thereafter. Examples of environmental stress include drying, high temperature stress, and the like involving DREB2A. For example, resistance to drought stress can be evaluated by withholding water for 2-4 weeks after growing plants for a certain length of time (for example, 2-3 weeks) on GM agar medium, then transplanting to soil containing vermiculite, pearlite, and the like and growing for a certain length of time (for example, 2 days to one week), then growing under ordinary conditions for 1-2 weeks and studying their survival. Resistance to high temperature stress can be evaluated by leaving plants grown for a certain length of time (for example 6-8 days) on filter paper soaked with GM liquid medium for one hour at 42° C., then growing under ordinary conditions for from four days to two weeks and studying their survival.

Those skilled in the art who have received the above explanation can implement the present invention adequately. Examples are given below for the sake of further explanation. Therefore, the present invention is not limited to these examples. Furthermore, the nucleotide sequences in this specification are listed in the direction from 5′ to 3′ unless stated otherwise.

### Example 1

### Interaction Analysis by a Yeast Two-Hybrid System

Interaction analysis of DREB2A and NF-YC10 by a yeast two-hybrid system followed the protocol of Clontech's Matchmaker System (http://www.clontech.com/JP/Products/Protein_Interactions_and_Profiling/Yeast_Two-Hybrid/Matchmaker_Gold_Yeast_Two-Hybrid_System?sitex=10025:22372:US).

Specifically, a pGBKT7 plasmid to which a base sequence of bases 1-615 encoding a region (1-205 a.a) excluding the transcriptional activation domain of the C terminal side in DREB2A cDNA had been ligated (transferred from Feng Qin Ph.D., the Japan International Research Center for Agricultural Sciences (Japan)) was used as the bait gene vector. A prey gene vector was also produced by PCR amplification of full-length cDNA of Arabidopsis thaliana NF-YC10 by the following primer pair and introduction of the amplified sequence obtained at the ClaI/XhoI site of pGADT7.

[Chemical Formula 6]
f o r w a r d:
(SEQ ID NO: 18)
5′ - C C A T C G A T A C A T G G T G T C G T C A A
A G A A - 3′
r e v e r s e:
(SEQ ID NO: 19)
5′ - A T C T C G A G T C A G C C T G C A T C T G T
C A T - 3′

The above bait vector and prey vector were introduced into yeast AH109. The existence of interaction of DREB2A and Arabidopsis thaliana NF-YC10 was confirmed by the growth of the yeast into which both vectors had been introduced on SD/-Leu/-Trp/-His/-Ade/3-AT (QDO) agar medium (see FIG. 5). This result was confirmed not to be a false positive by the fact that a strain having a pGBKT7 plasmid into which no DREB2A gene (bases 1-615 of the coding sequence) had been inserted introduced together with the above prey vector did not grow.

### Example 2

### Interaction Analysis by Transient Expression Using Arabidopsis thaliana Protoplasts

Transient expression analysis using protoplasts from Arabidopsis thaliana mesophyll cells was conducted in accordance with the method of Yoo et al. [Yoo et al., Nat. Protoc., Vol. 2, pp. 1565-1572 (2004)]. To offer a brief explanation, the gene was introduced into the protoplast; after standing for 14 hours, MG132, a proteasome inhibitor, was added to make a final concentration of 50 μM to stabilize the DREB2A in the plant. After standing for another 4 hours, the YFP fluorescence was examined.

Specifically, two plasmids containing DREB2A and Arabidopsis thaliana NF-YC10 were produced by amplifying full-length cDNA of DREB2A and full-length cDNA of Arabidopsis thaliana NF-YC10 by PCR and attaching each to either pBI221 [Qin et al., Plant Cell, Vol. 20, pp. 1693-1707 (2008)] which is an expression vector containing cDNA encoding the N terminal side or C terminal side of YFP, and a transient expression analysis study of both plasmids was conducted. Furthermore, one transferred from Feng Qin Ph.D. (Japan International Research Center for Agricultural Sciences, Japan) was used as the DREB2A full-length cDNA-ligated pBI221 plasmid. Full-length cDNA of Arabidopsis thaliana NF-YC10 was produced by amplification by PCR using the following primer pair and introduction at the XbaI/ClaI site of the above expression vector.

[Chemical Formula 7]
f o r w a r d:
(SEQ ID NO: 20)
5′ - C G T C T A G A A T G G T G T C G T C A A A
G A A - 3′
r e v e r s e:
(SEQ ID NO: 21)
5′ - A T A T C G A T T C C G C C T G C A T C T G T
C A T - 3′

The existence of interaction of DREB2A and Arabidopsis thaliana NF-YC10 was confirmed by the fact that a signal was generated only in the protoplasts from Arabidopsis thaliana mesophyll cells having both plasmids introduced (see FIG. 6). Furthermore, in the figure, bZIP63 is a positive control known to form a homodimer. CFP is introduced simultaneously to confirm proper gene introduction.

### Example 3

### Phenotype Analysis of NF-YC10-Overexpressing Arabidopsis thaliana

The ability of Arabidopsis thaliana NF-YC10 and DREB2A to interact was confirmed by the above study. The possibility that the growth rate, dwarfing, and other such phenotypes of the transformed plant are changed by overexpression of Arabidopsis thaliana NF-YC10 was therefore examined.

Transformed plants that express cDNA of Arabidopsis thaliana NF-YC10 under the control of a cauliflower mosaic virus 35S promoter were produced for this purpose. Specifically, full-length cDNA of Arabidopsis thaliana NF-YC10 was amplified by PCR using the following primers and introduced at the XbaI/XhoI site of a pGKX vector. The pGKX vector has an enhancer E12, CaMV35S promoter, Ω sequence, multicloning site, and Nos-T sequence inserted into pGreen0029 and is described in the paper of Yoshida et al. [Yoshida et al., Biochem. Biophys. Res. Commun., Vol. 368, pp. 515-521 (2008)]. The transformed vector obtained was named pGreen-NF-YC10.

[Chemical Formula 8]
f o r w a r d:
(SEQ ID NO: 22)
5′ - G C T C T A G A G A T G G T G T C G T C A A A
G A A - 3′
r e v e r s e:
(SEQ ID NO: 23)
5′ - A T C T C G A G T C A G C C T G C A T C T G T
C A T - 3′

Transformed plants that overexpress Arabidopsis thaliana NF-YC10 under the control of a CaMV 35S promoter were produced by introducing the above transformed vector pGreen-NF-YC10 into Arabidopsis thaliana (Columbia) by the floral dip method using Agrobacterium tumefaciens C58 [Clough et al., Plant J., Vol. 16, pp. 735-743 (1998)]. The expression of the transgene NF-YC10 was then analyzed in a stress-free state in 20 independent lines of transformed plants. Three separate lines having a high expression level of the introduced Arabidopsis thaliana NF-YC10 were selected for phenotype analysis. FIG. 7 shows the results of northern analysis of these three lines (35S:NF-YC10-a, 35S:NF-YC10-b, and 35S:NF-YC10-c) and a control transformed by only an empty vector (VC; vector control). Each lane is the result of phoresis of 10 μg of total RNA. The lower row is the control stained by ethidium bromide.

The phenotype of the three lines of transformed plants selected as described above was analyzed by growing the plants on GMK agar medium for 2-3 weeks under lighting conditions of 16 hours light/8 hours dark (40±10 μmol photons/m²/s) at 22±1° C. according to the method of Osakabe et al. [Osakabe et al., Plant Cell, Vol. 17, pp. 1105-1119 (2005)]. Furthermore, 1% sucrose was added to the GMK agar medium. The plants were also transplanted to pots as needed, and transformed plants were grown under the same conditions in soil such as vermiculite, pearlite, or the like. The photographs of FIG. 8A show the growth state of the three lines of transformed plants selected as described above after being grown for 16 days from sowing on 1% sucrose-containing GMK medium. FIG. 8B shows photographs taken after transplanting to pots and growing for another two weeks after growing the three lines of transformed plants for two weeks from sowing on 1% sucrose-containing GMK medium. The results obtained by measuring the maximum radius of the rosette leaves and the inflorescence length of each plant are shown in FIGS. 9A and 9B. Since no significant differences were seen in the growth of the plants of the present invention that overexpressed Arabidopsis thaliana NF-YC10 and the vector control plants in these studies, overexpression of the gene of the present invention was confirmed to exert no substantial effect on plant growth.

### Example 4

### Expression Analysis of Downstream Genes of the DREB2A Gene in NF-YC10-Overexpressing Arabidopsis thaliana

Changes in the transcription of downstream genes of DREB2A, such as RD29A and HsfA3, due to the interactivity of Arabidopsis thaliana NF-YC10 and DREB2A were studied. Transcription of these downstream genes was studied in particular under drought stress and high temperature stress conditions where DREB2A is thought to function. The expression of RD29A and RD29B having only drought-inducibility and HsfA3 and At1g75860 having high temperature-inducibility was studied as downstream genes.

Specifically, the three lines of transformed plants selected in Example 3 and vector control plants transformed by an empty vector were tested. Drought stress was applied by removing plants grown for a certain length of time (2-3 weeks) on GM agar medium and leaving them for 1-24 hours on filter paper. High temperature stress was applied by leaving plants grown for a certain length of time (2-3 weeks) on GM agar medium for 1-24 hours at 37° C. on GM agar medium. Eight plants were taken as one sample in RNA extraction for biological replication. The total RNA from the plant was prepared, electrophoresed, and changes in the expression pattern were analyzed by the northern method using DNA fragments of the gene the expression of which was to be confirmed as a probe. Total RNA extraction from the plants and RNA gel blot analysis were performed by the method of Satoh et al. [Satoh et al., Plant Cell Physiol., Vol. 45, pp. 309-317 (2004)] using a Sakemaster crusher (Bio Medical Science, Tokyo, Japan). The probe in RNA gel blot analysis was prepared according to the method of Maruyama et al. [Maruyama et al., Plant J., Vol. 38, pp. 982-993 (2004)].

FIG. 10 shows the results obtained when stress was applied for 1 to 10 hours. Changes in expression of RD29A and RD29B could not be confirmed during drought or high temperature stress. However, expression of HsfA3 and At1g75860 rose significantly in two lines of transformants of the present invention in comparison to the vector control with five hours of high temperature stress treatment.

### Example 5

### Microarray Analysis of NF-YC10-Overexpressing Arabidopsis thaliana

Microarray analysis was carried out after high temperature stress using two separate lines of transformants having marked changes in expression levels of HsfA3 and At1g75860, downstream genes of DREB2A, within the above NF-YC10-overexpressing Arabidopsis thaliana. A Custom Gene Expression Microarray, 4×44K, version 3 (Agilent Technologies, Palo Alto, Calif., USA) capable of examining the expression profile of all Arabidopsis thaliana genes was used as the microarray.

Specifically, the two separate lines of transformants described above and control vector plants that had been grown for two weeks by GM agar medium were collected after five hours of treatment at 37° C. Eight plants were taken as one sample in RNA extraction for biological replication. The total RNA was extracted by RNAiso (Takara) and used in the preparation of cDNA probes labeled by Cy5 and Cy3. The entire microarray study, including data analysis, was conducted in accordance with the product protocol (http://www.genomics.agilent.com/GenericA.aspx?pagetype=Custom &subpagetype=Custom&pageid=2018). A study was conducted to confirm that the same results were obtained even when the dye (Cy5 and Cy3) was exchanged to evaluate the reproducibility of microarray analysis. Assay was performed by identifying each spot on the array using feature extraction and image analysis software (version A.6.1.1; Agilent Technologies), and normalization was carried out by the Lowess method.

Fifteen genes induced at an expression level twice or more that of the vector control plants were discovered at a signal value of 2000 or higher and an FDR (false discovery rate) of 0.01 or less with five hours of treatment at 37° C. by the above microarray analysis (FIG. 11). Five of these 15 genes were HSP (heat shock protein) and three were HSF (heat shock factor). In addition, of the five HSP, three were genes the expression of which also rose in microarray analysis of DREB2A CA-overexpressers [Sakuma et al., Proc. Natl. Acad. Sci., Vol. 103, pp. 18822-18827 (2006)].

### Example 6

### Test of the High Temperature Stress and Drought Stress Resistance of NR-YC10-Overexpressing Arabidopsis thaliana

HSP and HSF among the stress response genes are thought to be involved with high temperature stress resistance [Montero-Barrientos et al., J. Plant Physiol., Vol. 167, pp. 659-665 (2010); Yoshida et al., Biochem. Biophys. Res. Commun., Vol. 368, pp. 515-521 (2008)]. As in Example 5 above, eight of the 15 genes the expression of which was elevated under high temperature stress were HSP or HSF. Arabidopsis thaliana NF-YC10-overexpressing plants were therefore expected to have improved high temperature stress resistance. To confirm this, the resistance of the three separate lines selected above in Example 3 to high temperature stress was studied. Resistance to drought stress, in which DREB2A is assumed to be involved, was also studied.

Specifically, as regards drought stress, plants that had been grown for two weeks after sowing under lighting conditions of 16 hours light/8 hours dark (50±10 μmol photons/m²/s) at 22±1° C. on GM agar medium were transplanted to pots containing soil such as vermiculite, pearlite, or the like and grown for one week. Drought stress was then applied by withholding water from these plants for three weeks. After the application of this stress, the plants were again watered and grown for another two weeks, and their survival or death was determined by the color of the plant. Furthermore, plants of about the same size were used in the study to minimize the effects of plant size.

As regards high temperature stress, plants that had been grown for one week after sowing on filter paper soaked with GM liquid medium were treated for 50 minutes at 45° C. according to the method of Sakuma et al. [Sakuma et al., Proc. Natl. Acad. Sci., Vol. 103, pp. 18822-18827 (2006)]. Their condition was examined after growing for 10 days thereafter under ordinary conditions. More specifically, two sheets of filter paper (manufactured by Advantec) 84 mm in diameter were placed in a plastic petri dish (90 diameter×20 mm) and soaked with 4 mL of liquid GM medium. Sterilized seeds of the above three lines of transformants of the present invention were sown on top. Comparative control plants were also sown in the same dish. The perimeter of the dish was sealed by surgical tape (manufactured by 3M Health Care), and the dishes were placed on top of a 130 mm×130 mm×50 mm freeze box (manufactured by Assist Inc.) positioned in a hybridization incubator (instrument name HB-80: manufactured by Taitec) set at 45° C. seven days after sowing. After standing on the freeze box for 50 minutes, the dishes were returned to the 22° C. incubator and grown for 10 days. All of the studies were repeated three times or more and at least 40 plants of the three lines were used in each study.

FIG. 12 shows the results of the above high temperature stress resistance test. The numbers of denominator and numerator in parentheses in the figure represent the number of plants used in the study and the number of surviving plants, respectively. The survival rate (%) is also shown in the figure. While more than half the vector control plants died in this high temperature stress test, virtually all of the Arabidopsis thaliana NF-YC10-overexpressing plants of the present invention, especially lines 35S:NF-YC10-b and 35S:NF-YC10-c, stayed healthy. Therefore, expressing Arabidopsis thaliana NF-YC10 was shown to improve the high temperature stress resistance of plants. On the other hand, most of the plants died in the drought stress resistance test in this example, and no significant difference in survival rate could be found between the vector control and the NF-YC10-overexpressing plants.

### Example 7

### Phylogenetic Tree Analysis of NF-YC10 Homologous Genes in Arabidopsis thaliana and Other Plants and Animals

Alignment was produced by a Clustal W program from the sequence of the H2A domain, which is the conserved region of NF-YC family genes. The variables were set so that: gap open penalty=10.00, gap extension penalty=0.1. Furthermore, alignment was fine-tuned manually in the end. A phylogenetic tree was produced by the neighbor-joining method using MEGA software (version 4.1) in accordance with the method of Fujita et al. [Fujita et al., Plant J., Vol. 39, pp. 863-876 (2004)]. The monophyletic group reliability was calculated by bootstrap analysis (1000 iterations). FIG. 13 shows the homologous genes corresponding to the dicotyledonous Arabidopsis thaliana NF-YC10 and the designations assigned by the present inventors in Glycine max, the monocotyledonous Oryza sativa, the moss Physcomitrella patens, and the blue-green algae Chlamydomonas and Volvox. FIG. 14 shows the relationships of these genes and human, mouse, and yeast NF-YC family genes by a phylogenetic tree. The black dots in the figure show a bootstrap value of 50 or higher. It is apparent from this phylogenetic tree that Arabidopsis thaliana NF-YC10 is a phylogenetically located away from other related genes and that Arabidopsis thaliana NF-YC10 homologous genes in Glycine max, Oryza sativa, and Physcomitrella patens as well are similarly phylogenietically located away from other related genes. FIG. 15 shows a comparison of the amino acid sequences of the conserved regions of Arabidopsis thaliana and Arabidopsis thaliana NF-YC10 homologous genes in Oryza sativa, Glycine max, and Physcomitrella patens.

### Example 8

### Interaction Analysis by a Yeast Two-Hybrid System in Other Plant Genes

The Oryza sativa OsDREB2B2 gene is known to be a homologous gene of Arabidopsis thaliana DREB2A (Non-patent Reference 7). The Glycine max GmDREB2A; 2 gene is also known to be a homologous gene of Arabidopsis thaliana DREB2A (Non-patent Reference 8). This example shows that Arabidopsis thaliana NF-YC10 protein can also interact with DREB2A homologous proteins of other plants. In other words, it shows that the conserved region in the Arabidopsis thaliana NF-YC10 homologous protein family plays a decisive role in interactions of NF-YC10 homologous proteins and DREB2A homologous proteins.

A base sequence encoding this region (1-146 a.a.) of Oryza sativa OsDREB2B2 or a base sequence encoding this region (1-137 a.a.) of Glycine max GmDREB2A; 2 was ligated instead of the base sequence encoding a region excluding the transcriptional activation domain on the C terminal side of Arabidopsis thaliana DREB2A and used as the bait gene vector in the yeast two-hybrid system protocol used in Example 1. Specifically, a base sequence encoding OsDREB2B2 (1-146 a.a.) was amplified by PCR from a nucleotide containing the full-length cDNA of OsDREB2B2 (SEQ ID NO: 29) by the following primers (SEQ ID NOS: 30 and 31). Alternatively, a base sequence encoding GmDREB2A; 2 (1-137 a.a.) was amplified by PCR from a nucleotide containing the full-length cDNA of GmDREB2A; 2 (SEQ ID NO: 32) by the following primers (SEQ ID NOS: 33 and 34). These sequences were substituted for the Arabidopsis thaliana DREB2A sequence at the EcoRI/BamHI site of the pGBKT7 plasmid of Example 1 and taken as bait gene vectors. Aside from this, an Arabidopsis thaliana NF-YC10 full-length cDNA-ligated prey vector was introduced together with the bait vector into yeast AH109 and cultured in the same way as in Example 1.

[Chemical Formula 9]
O s D R E B 2 B 2:
(SEQ ID NO: 29)
G C C T T T C C T T C C G A T C T C T C T C C C T C T C
T C T C T T C T T C T T C T T C T T C C T T C C C T C T C A A C C C G A C G
A C C C A C G C G A A G C G A A C T C T C G C G C G A G A C G A G A G T A G
T A A A C C C T A G A A A C C T A G A G G A G A T C C C C A C C A C C A C C
A T G A C G G T G G A T C A G A G G A C G A C G G C G A A G G C G A T C A T
G C C G C C G G T G G A G A T G C C G C C C G T C C A G C C C G G A A G G A
A A A A G C G A C C A C G G A G A T C A C G C G A T G G A C C T A C T T C A
G T T G C A G A G A C C A T C A A G C G G T G G G C C G A G C T C A A C A A
T C A G C A G G A G C T T G A T C C A C A G G G T C C A A A G A A G G C A A
G G A A G G C A C C T G C A A A G G G T T C A A A G A A G G G C T G C A T G
A A G G G G A A A G G A G G A C C G G A G A A T A C A C G T T G T G A C T T
C C G T G G T G T G A G G C A A C G T A C C T G G G G C A A G T G G G T T G
C T G A A A T T C G G G A G C C G A A T C A G C A A A G T A G A C T C T G G
T T G G G G A C C T T C C C A A C T G C C G A A G C T G C A G C T T G T G C
T T A T G A C G A G G C A G C C A G A G C A A T G T A T G G T C C A A T G G
C T C G C A C T A A T T T T G G C C A C C A T C A T G C C C C T G C T G C T
T C C G T T C A G G T T G C A C T A G C A G C T G T C A A A T G T G C T T T
A C C T G G T G G T G G C T T A A C A G C A A G C A A G T C T A G A A C A T
C C A C T C A G G G T G C A T C A G C A G A T G T T C A A G A T G T T T T A
A C T G G T G G C T T A T C A G C A T G C G A G T C C A C T A C A A C A A C
A A T T A A T A A T C A A T C T G A T G T C G T C T C T A C C T T A C A T A
A G C C A G A A G A G G T T T C T G A G A T C T C T A G T C C A C T G A G A
G C T C C A C C A G C T G T C C T G G A A G A T G G T T C T A A T G A A G A
C A A G G C T G A A T C G G T T A C C T A T G A T G A G A A C A T T G T C A
G C C A G C A G C G T G C C C C T C C T G A A G C C G A G G C T A G T A A T
G G A A G A G G C G A G G A G G T C T T T G A G C C T C T G G A A C C T A T
T G C C A G C C T A C C A G A G G A C C A A G G A G A T T A T T G T T T T G
A T A T T G A T G A G A T G C T G A G A A T G A T G G A A G C T G A C C C T
A C G A A C G A G G G T T T G T G G A A A G G C G A C A A A G A T G G A T C
A G A C G C C A T C C T G G A G C T T G G C C A G G A T G A A C C T T T C T
A C T A C G A A G G G G T T G A T C C A G G C A T G C T G G A C A A C T T G
C T C A G G T C T G A T G A G C C A G C A T G G T T A T T G G C A G A T C C
T G C G A T G T T C A T C T C C G G T G G C T T C G A A G A T G A C T C T C
A G T T C T T T G A G G G C T T G T G A T T T C C C C T T G G C G G C A G C
C G G C C A T A C T A A A A T T T T C T G G T G C T T T G G T C G G C T A G
C T C C T G C A C A T C G C C C T C A G G A T C A G C A A G A G A A A C A C
T G G A C C G G A T T G G G T T C G T T G G T G G A A C T G G A T G A G C A
T C T A G T A G C T A A G G A A A A A A G A T C C T T T T A T T T A G T T C
T G T A G G C A A T G G A A C T C C T T G A G A A C T C C G T T T C A G T G
T T T G T T A A T T T G A T A A C G C T T G C T T G T T T G T G T G T G T A
T A T C G A T C T C T T T T G A A G C A A T G A G A A A A A A A A A A G G A
C T G A A G A A A A T G T G T A T A T A T T C C A A G C G T T C T T C A G C
C T T T C T T A G C C T T C A T A T T T T A C C T A T G C A C G T G G G A T
G T T G C A G T T T T A G A G C T T G T G A G C C T T C T C T A A A A C C G
G G G A T T A A A A T G C G A C T A G G C A C G A T A T G T T C A A T C T A
A A C C G A A C T C C C T A G G G T G T A T
[Chemical Formula 10]
f o r w a r d:
(SEQ ID NO: 30)
5′ - T A G A A T T C A T G A C G G T G G A T C A G A G G A C G-3′
[Chemical Formula 11]
r e v e r s e:
(SEQ ID NO: 31)
5′ - A T G G A T C C G G C C A A A A T T A G T G C G A G C - 3′
[Chemical Formula 12]
G m D R E B 2 A; 2:
(SEQ ID NO: 32)
A G A G A T T T T T C T G A A T C C G C T A T A G C
C A T A A C T C T T C A C G A A C A A G A A C T C T A C T A T T A C T A T T
A A T C A A C C A A A A T C T C T C T T C A C T C C A A A C A G A A C A C A
C T A G C G A G A A A A A A A G T G A T A A G C C C A A A A A C T C T G C G
T T C T C T C A C A A A T T A A A C A G C G T C A C T A T C G C A T A G A T
T G T G A A T T C A G T G A T T G A G T T T T G C G G T G T A C T G T G T T
G C G A A G T C T G T G T A T C A G A T T T G T G G A C A T G G G T G C T T
A T G A T C A A G T T T C T C T T A A G C C A T T G G A T T C T T C T A G A
A A G A G G A A A A G T A G G A G C A G A G G G T A T G G G A C T G G A T C
C G T G G C T G A G A C T A T T G C A A A G T G G A A G G A A T A C A A C G
A A C A T C T T T A T T C T G G C A A A G A T G A T A G T A G A A C A A C T
C G A A A G G C A C C G G C T A A A G G T T C G A A G A A A G G G T G C A T
G A A A G G G A A G G G A G G A C C T C A A A A C T C T C A G T G T A A C T
A C A G A G G A G T T A G G C A G A G G A C A T G G G G G A A A T G G G T T
G G T G A G A T T A G G G A G C C C A A T A G A G G A A G C A G G C T T T G
G T T G G G T A C C T T C T C T T C T G C C C A G G A A G C T G C T C T T G
C C T A T G A T G A A G C T G C T A G A G C T A T G T A T G G T C C T T G T
G C A C G C C T C A A T T T T C C C G G C A T C A C A G A T T A T G C T T C
T T T T A A G G A A T C G T T G A A G G A A T C T C C G A T G G C C G C A T
C G T C C T C T T G T T C T T C G G C A G A A A C T G C A A C A T C T G A C
A C T A C T A C T A C A T C C A A C C A A T C G G A G G T T T G T G C A G C
T G A G G A T G T T A A G G A G A A T C C T C G A C T T G T C A A T G T G A
A T G A T A A G G T T A A C G A T T G T C A T A A G G C T T A T G A A G C T
G C C T C A C C A A C T A G C A G A A T G A A G C A A G A G C C T A A G G A
T G A G G C T G T G G A T C A C A T G G T C C C C G G G G C T G G G A A A A
T T C T A G A T G T C A G A C C A G A A G G A A C A C A T G A T G C C G G G
C A G G T T G C A G A G G A T G T A A A C A A A G A T C A G A T G G A C T T
G C C A T G G A T T G A T G G C T T T G A T T T T A G T G A C A A T T A C T
T G A A C A G G T T T T C C A C G G A T G A G T T A T T T C A G G T G G A T
G A A C T T T T G G G G C T T A T A G A T A A T A A C C C A A T T G A T G A
G T C T G C G T T G A T G C A A A G T T T G G A T T T T G G A C A A A T G G
G T T T T C C T G G A G A T G G T A A T C C T C A G G T G G A T G A T A C G
C T T T C A A G C T T T A T T T A T C A G T T G C A A A A T C C A G A T G C
C A A G T T G T T G G G A A G T T T G C C C C A T A T G G A G C A G A C A C
C T T C A G G T T T T G A T T A T G G A T T A G A T T T C T T A A A A A C A
G T G G A G T C A G G G G A T T A T A A T G G T G G A G G G G A A G A A C C
G C G A T T T C T T A A T T T G G A T G A T G A T C T G A A C C C T G A T T
C A A A G G G C A T G C A A G C A A G G A A G G A T G A C T A G A G A A G G
C G A C G T G C A T A A G T C T A T C A T C T G C C T C A T T T T C A A C T
G G T T C G A G C A T C T G C T A G T A A T C T G T C T C T T A G G T T G T
T G T C C C C T T T T T A G C T A T A T A C A G G T G C A T A A G A G G A A
T A C A A C T A T A C A A C T A A T A C A A G A A A T T T G A T T T G T T T
A T G T T C T T T T A A T A T G C T A A T T C T C T G T A A G A T T T T T T
A A A A T G G A G A A T T T A G C T G T G A C A A T A T T T G T T A A T T C
T T T T T A C T T A C A T G T T T T T T G G G A T T C A A A T T G G A C T G
C C T T T A A C T A C A T A G G T G G A G C T G A G G A G T A G A C T G T T
T G A A G T C G T T T G G C T G A C T A T A G T T G A G C A C T G A T T T G
G A T A C A A A A T T T C T T T G T T A T G T A C C A T G G A G A A C T A T
T A T A T C T C G A G T A T A T T A T A T C G T T G C T C A C T T T T T G T
G T A T A A A A A C T G A A C A A G T A G T G G A A T G T A T A T A T A T A
T A A T A A C T A T T C T
[Chemical Formula 13]
(SEQ ID NO: 33)
5′ - G C G A A T T C A T G G G T G C T T A T G A T C A A G T TTC-3′
[Chemical Formula 14]
(SEQ ID NO: 34)
5′ - A T G G A T C C T T T G G G A A A A T T G A G G C G T G-3′

The presence of interaction of Oryza sativa OsDREB2B2 and Glycine max GmDREB2A;2 with Arabidopsis thaliana NF-YC10 was confirmed by the growth of yeast with both vectors introduced on SD/-Leu/-Trp/-His/-Ade/3-AT (QDO) agar medium (see FIG. 16).

### Example 9

### Interaction Analysis by Transient Expression Using Arabidopsis thaliana Protoplasts in Other Plant Genes

In addition to the above Example 8, the existence of the above interaction was confirmed by repeating the study of Example 2 using the Oryza sativa OsDREB2B2 gene and the Glycine max GmDREB2A;2 gene. Specifically, OsDREB2B2 full-length cDNA and GmDREB2A; 2 full-length cDNA were ligated to pBI221 instead of the Arabidopsis thaliana DREB2A full-length cDNA in Example 2. Furthermore, OsDREB2B2 full-length cDNA was amplified by PCR by the following primers (SEQ ID NOS: 35 and 36). GmDREB2A; 2 full-length cDNA was amplified by PCR by the following primers (SEQ ID NOS: 37 and 38). These cDNA were substituted for the Arabidopsis thaliana DREB2A cDNA at the ClaI/XhoI site of the pBI221 plasmid of Example 2. Aside from this, the same study as in Example 2 was repeated.

[Chemical Formula 15]
f o r w a r d:
(SEQ ID NO: 35)
5′ - T A A T C G A T A T G A C G G T G G A T C A G
A G G A CG-3′
r e v e r s e:
(SEQ ID NO: 36)
5′ - A T C T C G A G T C C C A A G C C C T C A A A
G A A C TG-3′
f o r w a r d:
(SEQ ID NO: 37)
5′ - G C A T C G A T A T G G G T G C T T A T G A T
C A A G TTTC-3′
r e v e r s e:
(SEQ ID NO: 38)
5′ - A T C T C G A G C T A G C C A C C C T T C C T
T G C T T-3′

The presence of interaction of Oryza sativa OsDREB2B2 and Glycine max GmDREB2A;2 with Arabidopsis thaliana NF-YC10 was confirmed by the fact that a signal was generated only in the protoplasts from Arabidopsis thaliana mesophyll cells with both plasmids introduced (see FIG. 17).

### INDUSTRIAL APPLICABILITY

The present invention can be utilized in foods, feeds, horticultural crops, and other such agricultural production. It can also be utilized in the seed industry for production of these crops. The transformed plants of the present invention can also be utilized in research and development in the plant biotechnology field.

1. A transformed plant showing improved resistance to environmental stress that overexpresses a gene containing a nucleotide sequence selected from the group consisting of:
(1) a nucleotide sequence encoding a protein comprising an amino acid sequence shown by SEQ ID NO: 2, 4, 6, or 8;
(2) a nucleotide sequence encoding a protein having 60% or higher sequence homology to the amino acid sequence shown by SEQ ID NO: 2, 4, 6, or 8 and having an ability to bind to DREB2A (dehydration responsive element binding protein 2A) protein; and
(3) a nucleotide sequence that hybridizes under stringent conditions with a nucleic acid comprising a nucleotide sequence complementary to a nucleotide sequence shown by SEQ ID NO: 1, 3, 5, 7, 14, 15, 16, or 17 and encodes a protein having the ability to bind to DREB2A protein.

2. The transformed plant according to claim 1, wherein the nucleotide sequence of (1) is a nucleotide sequence of a coding region of a nucleotide sequence shown by SEQ ID NO: 1, 3, 5, or 7.

3. The transformed plant according to claim 1, wherein the sequence homology in (2) is 80% or higher.

4. The transformed plant according to claim 1, wherein the environmental stress is high temperature stress.

5. The transformed plant according to claim 1, wherein the transformed plant is in a form of a seed, a seedling or a callus.

6-7. (canceled)

8. The transformed plant according claim 1, wherein the transformed plant is a dicotyledonous plant.

9. The transformed plant according to claim 1, wherein the transformed plant is a monocotyledonous plant.

10. A method for producing a transformed plant showing improved resistance to environmental stress, comprising:
i) a step for transforming a plant cell, wherein
a)the plant cell is transfected by an expression vector containing a nucleotide sequence selected from the following group to cause overexpression of the gene containing the nucleotide sequence in the cell:
(1) a nucleotide sequence encoding a protein comprising an amino acid sequence shown by SEQ ID NO: 2, 4, 6, or 8;
(2) a nucleotide sequence encoding a protein having 60% or higher sequence homology to the amino acid sequence shown by SEQ ID NO: 2, 4, 6, or 8 and having an ability to bind to DREB2A (dehydration responsive element binding protein 2A) protein; and
(3) a nucleotide sequence that hybridizes under stringent conditions with a nucleic acid comprising a nucleotide sequence complementary to a nucleotide sequence shown by SEQ ID NO: 1, 3, 5, 7, 14, 15, 16, or 17 and encodes a protein having the ability to bind to DREB2A protein; or
b) a control region of an endogenous gene containing a nucleotide sequence selected from the group of a) (1)-(3) above is replaced by an exogenous control element in the plant cell to cause overexpression of the gene in the cell; and
ii) a step for causing a growth of a transformed plant cell obtained in step i) above under conditions suited to regeneration of a plant from the cell to obtain a transformed plant.

11. The method for producing a transformed plant according to claim 10, wherein the nucleotide sequence of (1) is a nucleotide sequence of a coding region of a nucleotide sequence shown by SEQ ID NO: 1, 3, 5, or 7.

12. The method for producing a transformed plant according to claim 10, wherein the sequence homology in (2) is 80% or higher.

13. The method for producing a transformed plant according to claim 10, wherein the environmental stress is high temperature stress.

14. The method for producing a transformed plant according to claim 10, wherein the transformed plant is in a form of a seed, a seedling or a callus.

15-16. (canceled)

17. The method for producing a transformed plant according to claim 10, wherein the transformed plant is a dicotyledonous plant.

18. The method for producing a transformed plant according to claim 10, wherein the transformed plant is a monocotyledonous plant.

19. A method for improving the resistance of a plant to environmental stress, comprising:
a) transfecting a plant cell by an expression vector containing a nucleotide sequence selected from the following group and causing overexpression of the gene containing the nucleotide sequence in the cell:
(1) a nucleotide sequence encoding a protein consisting of an amino acid sequence shown by SEQ ID NO: 2, 4, 6, or 8;
(2) a nucleotide sequence encoding a protein having 60% or higher sequence homology to an amino acid sequence shown by SEQ ID NO: 2, 4, 6, or 8 and having the ability to bind to DREB2A (dehydration responsive element binding protein 2A) protein; and
(3) a nucleotide sequence encoding a protein that hybridizes under stringent conditions with a nucleic acid comprising a nucleotide sequence complementary to a nucleotide sequence shown by SEQ ID NO: 1, 3, 5, 7, 14, 15, 16, or 17 and having the ability to bind to DREB2A protein; or
b) replacing a control region of an endogenous gene containing a nucleotide sequence selected from the group of a) (1)-(3) above by an exogenous control element in a plant cell and causing overexpression of the gene in the cell.

20. The method according to claim 19 wherein the nucleotide sequence of (1) is a nucleotide sequence of a coding region of a nucleotide sequence shown by SEQ ID NO: 1, 3, 5, or 7.

21. The method according to claim 19 wherein the sequence homology in (2) is 80% or higher.

22. The method according to claim 19 wherein the environmental stress is high temperature stress.

23. The method according to claim 19 wherein the transformed plant is in the form of a seed, a seedling or a callus.

24-25. (canceled)

26. The method according to claim 19 wherein the plant is a dicotyledonous plant.

27. The method according to claim 19 wherein the plant is a monocotyledonous plant.
